(19) United States
(12) Patent Application Publication
(10) Pub. No.: US 2018/0016227 A1
(43) Pub. Date: Jan. 18, 2018

(54) PROCESS FOR THE SYNTHESIS OF DIFLUOROMETHYL ETHER-BASED COMPOUNDS

(71) Applicant: Trillium Therapeutics Inc., Mississauga (CA)

(72) Inventors: Abdelmalik Slassi, Mississauga (CA); Peter Dove, Burlington (CA)

(73) Assignee: Trillium Therapeutics Inc., Mississauga, ON (CA)

(21) Appl. No.: 15/544,939

(22) PCT Filed: Feb. 3, 2016
(86) PCT No.: PCT/CA2016/050095

§ 371 Date: Jul. 20, 2017

Related U.S. Application Data
(60) Provisional application No. 62/111,251, filed on Feb. 3, 2015.
(60) Provisional application No. 62/114,760, filed on Feb. 11, 2015.

(51) Int. Cl. C07C 269/06; C07D 265/30; C07D 211/46; C07D 207/12; C07C 303/30; C07D 207/08; C07D 205/04; C07C 327/06; C07D 317/22; C07D 211/22

(52) U.S. Cl. CPC C07C 269/06; C07D 317/22; C07D 265/30; C07D 211/46; C07D 211/22; C07C 303/30; C07D 207/08; C07D 205/04; C07C 327/06; C07D 207/12

(57) ABSTRACT

The present application relates to a novel process for the preparation of difluoromethyl ether-based derivatives from, for example, aliphatic and aromatic hydroxyl precursors, compositions comprising these compounds and their use, in particular as precursors for medicines for the treatment of diseases, disorders or conditions. In particular, the present application includes the process of preparing compounds of Formula (I), and compositions and uses thereof:

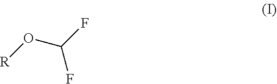

### CROSS-REFERENCE TO RELATED APPLICATIONS

The present application claims the benefit of priority from co-pending U.S. provisional patent application Ser. No. 62/111,251 filed on Feb. 3, 2015 and co-pending U.S. provisional patent application Ser. No. 62/114,760 filed on Feb. 11, 2015, the contents of both of which are incorporated herein by reference.

### FIELD

The present application includes a process for the preparation of difluoromethyl-ether derivatives from aliphatic and aromatic hydroxyl precursors.

### BACKGROUND

Fluorine has found interest in bioorganic and structural chemistry over the past decade and has become a useful feature in drug design. The small and highly electronegative fluorine atom can play a useful role in medicinal chemistry. Selective installation of fluorine into a therapeutic or diagnostic small molecule candidate can give a number of useful pharmacokinetic and/or physicochemical properties such as improved metabolic stability and enhanced membrane permeation. Increased binding affinity of fluorinated drug candidates to a target protein has also been documented in a number of cases. A further emerging application of the fluorine atom is the use of ¹⁸F as a radiolabel tracer atom in the sensitive technique of Positron Emission Tomography (PET) imaging.

Fluorine substitution has been investigated in drug research as a means of enhancing biological activity and/or increasing chemical and/or metabolic stability. Factors to be considered when synthesising fluorine-containing compounds include (a) the relatively small size of the fluorine atom (van der Waals radius of 1.47 Å), comparable to hydrogen (van der Waals radius of 1.20 Å), (b) the highly electron-withdrawing nature of fluorine, (c) the greater stability of the C—F bond compared to the C—H bond and (d) the greater lipophilicity of fluorine compared to hydrogen.

Despite the fact that fluorine is slightly larger than hydrogen, several studies have demonstrated that it is a reasonable hydrogen mimic and is often expected to cause minimal steric perturbations with respect to the compound's mode of binding to a receptor or enzyme [Annu. Rev. Pharmacol. Toxicol. 2001, 41, 443-470]. However, the introduction of a fluorine atom can significantly alter the physicochemical properties of the compound due to its high electronegativity. Therefore this type of modification can induce altered biological responses of the molecule.

The introduction of the fluorine atom into molecules brings about dramatic changes in the physical and chemical properties of the parent molecules, and sometimes results in the enhancement of pharmacokinetic properties and biological activities. The unique properties of the fluorine atom include its small size, low polarizability, high electronegativity and its ability to form strong bonds with carbon. Recently, bioactive compounds containing trifluoromethoxy, difluoromethoxy and fluoromethoxy groups have attracted great interest. Replacement of hydrogen atoms can sometimes result in improved thermal and metabolic stability. Improved metabolic stability is usually a desirable feature since the possibility exists that in vivo decomposition may produce toxic effects.

The geminal combination of an alkoxyl or aryloxy group with a fluorine atom offers the possibility of bonding/nonbonding resonance, which can be formally expressed by the superposition of a covalent and ionic limiting structure. This phenomenon, which reveal itself as a lengthening and weakening of the carbon-halogen bond and a shortening and strengthening of the carbon-oxygen bond is widely known as the generalized anomeric effect [Schlosser et al Chem. Rev. 2005, 105: 827-856].

Literature examples of difluoromethylation are shown in Schemes 1 and 2. The O-α,α-difluoro alkyl ethers can be prepared by electrophilic reactions of the appropriate alkoxide anion with chlorodifluoromethylation in the presence of base [Clark et al J. Am. Chem. Soc. 1955, 77: 6618; Miller et al J. Org. Chem. 1960, 25: 2009, Sharma et al J. Fluorine. Chem. 1988, 41: 247]; difluorocarbene [Naumann et al J. Fluorine. Chem. 1994, 67: 91; Naumann et al Liebigs. Ann. 1995, 1717-1719] and difluoromethylcarbocation equivalent [Uneyama et al Tetrahedron Lett. 1993, 34: 1311; Uneyama et al J. Org. Chem. 1995, 60: 370].

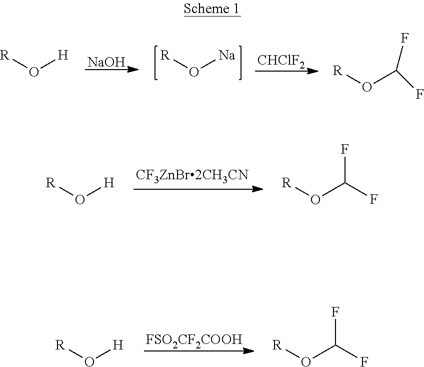

Alternatively, as shown in Scheme 2, the difluoromethyl ethers could also be accessible by sulfur tetrafluoride mediated fluorodeoxygenation of formates [Sheppard et al J. Org. Chem. 1964, 29: 1] or from the treatment of the alcohol with iododifluoromethyl phenyl sulphone to give the corresponding ether which can undergo reductive desulphonylation [Olah et al Org. Lett. 2005, 6: 4315].

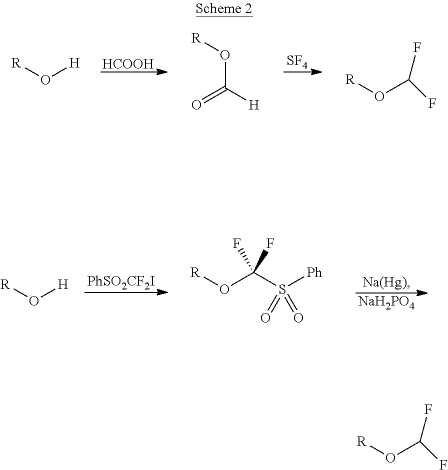

Difluoromethyl ethers are becoming increasingly prevalent in the pharmaceutical, (Modern Fluoroorganic Chemistry: Synthesis, Reactivity, Applications; Wiley-VCH: Weinheim, 2004) agrochemical, (Angew. Chem., Int. Ed. 2000, 39, 4216) and materials (Ferroelectronics 2002, 276, 83) industries. A number of previously developed chemistry routes have utilized chlorodifluoromethane (J. Org. Chem. 1960, 25, 2009; Tetrahedron Lett. 1961, 2, 43) a highly toxic chlorofluorocarbon (CFC) gas, as the source of the difluorocarbene intermediate. However, this reagent could not be used on a commercial scale. Other reagents used for the difluoromethylation include those derived from chlorodifluoroacetic acid, including the sodium salt and alkyl esters (WO199623754; Helv. Chim. Acta 2005, 88, 1040). Some of these reagents are bench-stable solids, are readily available in bulk and easier to handle than chlorodifluoromethane. However, the reactions must be carried out at elevated temperature, releases an equimolar amount of carbon dioxide, and produce unwanted byproducts such as double-addition and triple-addition adducts. Although a number of alternative reagents do exist for difluoromethyl ether formation, lack of commercial availability, high toxicity, and/or inadequate efficiency limit their use in the pharmaceutical industry. Furthermore, a thorough examination of the literature also suggests that difluoromethylation reactions are often plagued by low yields and/or limited scope (J. Org. Chem. 2006, 71, 9845; Chem. Commun. 2007, 5149; Tetrahedron Lett. 1981, 22, 323; J. Fluorine Chem. 1989, 44, 433).

### SUMMARY

Despite attempts to develop difluoromethylation procedures, the incompatibility of reagents with other functional groups, utilization of harsh conditions and low yield with increasing number of steps, the prior art methods are discouraging from a commercial point of view. The present application utilizes commercially viable synthesis of thioformyl esters to readily access highly functionalized difluoromethyl ethers. The present inventors have surprisingly found that the intermediates of the present application overcome the difficulties of the prior art and may be prepared and subsequently converted to difluoromethyl ethers in high yield and purity. This new method of difluoromethylation is safe and efficient and can be carried out on multikilogram scale.

Therefore one embodiment of the present application is an expedient commercially viable and useful process for the preparation of difluoromethyl ethers for the synthesis of pharmaceutically useful compounds.

Another embodiment of the present application is an expedient commercially viable and useful process for the preparation of difluoromethyl ethers of serines and threonines and other highly functionalized alcohols, via thioformyl intermediate precursors.

A further embodiment of the present application is an operationally simple route of synthesis for the production of difluoromethyl ethers in high yield and purity.

Accordingly, one aspect of the present application includes a process for the preparation of difluoromethyl ethers the process comprising:
a) reacting a suitable alcohol with Vilsmeier reagent, followed by a sulfurating reagent under conditions to provide a thioformyl ester; and
b) reacting the thioformyl ester of step (a) with 2,2-difluoro-1,3-dimethylimidazolidine under conditions to provide the difluoromethyl ether.

Another aspect of the present application includes a process for the preparation of difluoromethyl ethers of Formula (I) or pharmaceutically acceptable salts, solvates and/or prodrug thereof:

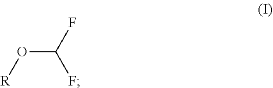

the process comprising:
   a) reacting a compound of Formula (II) with Vilsmeier reagent followed by a sulfurating reagent under conditions to provide the compound of Formula (III):

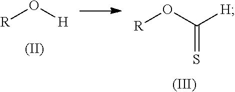

and
b) reacting a compound of Formula (III) with 2,2-difluoro-1,3-dimethylimidazolidine under conditions to provide the compound of Formula (I):

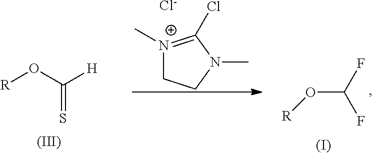

wherein
R is selected from D/L-amino acids, C₁₋₁₀alkyl, C₂₋₁₀alkenyl, C₂₋₁₀alkynyl, C₁₋₁₀haloalkyl, C₁₋₁₀cyanoalkyl, C₁₋₁₀alkoxy, C₂₋₁₀alkenyloxy, C₂₋₁₀alkynyloxy, C₃₋₁₀cycloalkyl, heterocycloalkyl, aryl, heteroaryl, C₁₋₆alkylene-O—C₁₋₆alkyl, C₁₋₆alkylene-O—C₁₋₆haloalkyl, C₂₋₆alkenylene-O—C₁₋₆haloalkyl, C₂₋₆alkynylene-O—C₁₋₆haloalkyl, C₁₋₆alkylene-C₃₋₈cycloalkyl, C₁₋₆alkylene-heterocycloalkyl, C₁₋₆alkylene-aryl, C₁₋₆alkylene-heteroaryl, C₁₋₁₀alkyl-C(O)R¹, C₂₋₁₀alkenyl-C(O)R¹, C₂₋₁₀alkynyl-C(O)R¹, C₁₋₁₀haloalkyl-C(O)R¹, C₁₋₁₀cyanoalkyl-C(O)R¹, C₁₋₁₀alkoxy-C(O)R¹, C₂₋₁₀alkenyloxy-C(O)R¹, C₃₋₁₀cycloalkyl-C(O)R¹, heterocycloalkyl-C(O)R¹, aryl-C(O)R¹, heteroaryl-C(O)R¹, C₁₋₆alkylene-O—C₁₋₆alkyl-C(O)R¹, C₁₋₆alkylene-O—C₁₋₆haloalkyl-C(O)R¹, C₂₋₆alkenylene-O—C₁₋₆haloalkyl-C(O)R¹, C₂₋₆alkenylene-O—C₁₋₆haloalkyl-C(O)R¹, C₁₋₆alkylene-C₃₋₈cycloalkyl-C(O)R¹, C₁₋₆alkylene-heterocycloalkyl-C(O)R¹, C₁₋₆alkylene-aryl-C(O)R¹, C₁₋₆alkylene-heteroaryl-C(O)R¹, C₁₋₁₀alkyl-OC(O)R¹, C₂₋₁₀alkenyl-OC(O)R¹, C₂₋₁₀alkynyl-OC(O)R¹, C₁₋₁₀haloalkyl-OC(O)R¹, C₁₋₁₀cyanoalkyl-OC(O)R¹, C₁₋₁₀alkoxy-OC(O)R¹, C₂₋₁₀alkenyloxy-OC(O)R¹, C₃₋₁₀cycloalkyl-OC(O)R¹, heterocycloalkyl-OC(O)R¹, aryl-OC(O)R¹, heteroaryl-OC(O)R¹, C₁₋₆alkylene-O—C₁₋₆alkyl-OC(O)R¹, C₁₋₆alkylene-O—C₁₋₆haloalkyl-OC(O)R¹, C₂₋₆alkenylene-O—C₁₋₆haloalkyl-O—C(O)R¹, C₂₋₆alkenylene-O—C₁₋₆haloalkyl-O—C(O)R¹, C₁₋₆alkylene-C₃₋₁₀cycloalkyl-O—C(O)R¹, C₁₋₆alkylene-heterocycloalkyl-O—C(O)R¹, C₁₋₆alkylene-aryl-O—C(O)R¹, C₁₋₆alkylene-heteroaryl-O—C(O)R¹, C₁₋₁₀alkyl-C(O)OR¹, C₂₋₁₀alkenyl-C(O)OR¹, C₂₋₁₀alkynyl-C(O)OR¹, C₁₋₁₀haloalkyl-C(O)OR¹, C₁₋₁₀cyanoalkyl-C(O)OR¹, C₁₋₁₀alkoxy-C(O)OR¹, C₂₋₁₀alkenyloxy-C(O)OR¹, C₃₋₁₀cycloalkyl-C(O)OR¹, heterocycloalkyl-C(O)OR¹, aryl-C(O)OR¹, heteroaryl-C(O)OR¹, C₁₋₆alkylene-O—C₁₋₆alkyl-C(O)OR¹, C₁₋₆alkylene-O—C₁₋₆haloalkyl-C(O)OR¹, C₂₋₆alkenylene-O—C₁₋₆haloalkyl-C(O)OR¹, C₂₋₆alkenylene-O—C₁₋₆haloalkyl-C(O)OR¹, C₁₋₆alkylene-C₃₋₈cycloalkyl-C(O)OR¹, C₁₋₆alkylene-heterocycloalkyl-C(O)OR¹, C₁₋₆alkylene-aryl-C(O)OR¹, C₁₋₆alkylene-heteroaryl-C(O)OR¹, C₁₋₆alkylene-O—R¹, C₁₋₆alkylene-C(O)R¹, C₁₋₆alkylene-O—C(O)R¹, C₁₋₆alkylene-C(O)OR¹, C₁₋₆alkylene-O—C(O)OR¹, C₁₋₆alkyleneNR¹R², C₁₋₆alkylene-NR¹R¹, C₁₋₆alkylene-C(O)NR¹R², C₁₋₆alkylene-NR¹C(O)R², C₁₋₆alkylene-NR¹C(O)NR³R², C₁₋₆alkylene-S—R¹, C₁₋₆alkylene-S(O)R¹, C₁₋₆alkylene-SO₂R¹, C₁₋₆alkylene-SO₂NR¹R², C₁₋₆alkylene-NR¹SO₂R², C₁₋₆alkylene-NR³SO₂NR¹R², C(O)NR¹R² and C₁₋₆alkylene-NR¹C(O)OR², wherein R can be optionally substituted with C₁₋₄alkyl and any cyclic moiety is optionally fused to a further cyclic and heterocyclic moieties; and
R¹, R² and R³ are each independently selected from H, C₁₋₆alkyl, C₁₋₆haloalkyl, C₂₋₆alkenyl, C₂₋₆alkynyl, C₃₋₁₀cycloalkyl, C₁₋₆alkylene-C₃₋₁₀cycloalkyl, heterocycloalkyl, aryl, C₁₋₆alkylene-aryl, C₁₋₆alkylene-heterocycloalkyl, heteroaryl, and C₁₋₆alkylene-heteroaryl, wherein any cyclic or heterocyclic moiety is optionally fused to a further cyclic or heterocyclic moiety.

A further aspect of the present application includes a compound of Formula (I) or a pharmaceutically acceptable salt, solvate and/or prodrug thereof:

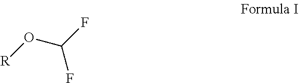

wherein:
R is selected from the group consisting of C₁₋₁₀alkyl, C₂₋₁₀alkenyl, C₂₋₁₀alkynyl, C₁₋₁₀haloalkyl, C₁₋₁₀cyanoalkyl, C₁₋₁₀alkoxy, C₂₋₁₀alkenyloxy, C₂₋₁₀alkynyloxy, C₃₋₁₀cycloalkyl, heterocycloalkyl, aryl, heteroaryl, C₁₋₆alkylene-O—C₁₋₆alkyl, C₁₋₆alkylene-O—C₁₋₆haloalkyl, C₂₋₆alkenylene-O—C₁₋₆haloalkyl, C₂₋₆alkynylene-O—C₁₋₆haloalkyl, C₁₋₆alkylene-C₃₋₈cycloalkyl, C₁₋₆alkylene-heterocycloalkyl, C₁₋₆alkylene-aryl, C₁₋₆alkylene-heteroaryl, C₁₋₁₀alkyl-C(O)R¹, C₂₋₁₀alkenyl-C(O)R¹, C₂₋₁₀alkynyl-C(O)R¹, C₁₋₁₀haloalkyl-C(O)R¹, C₁₋₁₀cyanoalkyl-C(O)R¹, C₁₋₁₀alkoxy-C(O)R¹, C₂₋₁₀alkenyloxy-C(O)R¹, C₃₋₁₀cycloalkyl-C(O)R¹, heterocycloalkyl-C(O)R¹, aryl-C(O)R¹, heteroaryl-C(O)R¹, C₁₋₆alkylene-O—C₁₋₆alkyl-C(O)R¹, C₁₋₆alkylene-O—C₁₋₆haloalkyl-C(O)R¹, C₂₋₆alkenylene-O—C₁₋₆haloalkyl-C(O)R¹, C₂₋₆alkenylene-O—C₁₋₆haloalkyl-C(O)R¹, C₁₋₆alkylene-C₃₋₈cycloalkyl-C(O)R¹, C₁₋₆alkylene-heterocycloalkyl-C(O)R¹, C₁₋₆alkylene-aryl-C(O)R¹, C₁₋₆alkylene-heteroaryl-C(O)R¹, C₁₋₁₀alkyl-OC(O)R¹, C₂₋₁₀alkenyl-OC(O)R¹, C₂₋₁₀alkynyl-OC(O)R¹, C₁₋₁₀haloalkyl-OC(O)R¹, C₁₋₁₀cyanoalkyl-OC(O)R¹, C₁₋₁₀alkoxy-OC(O)R¹, C₂₋₁₀alkenyloxy-OC(O)R¹, C₃₋₁₀cycloalkyl-OC(O)R¹, heterocycloalkyl-OC(O)R¹, aryl-OC(O)R¹, heteroaryl-OC(O)R¹, C₁₋₆alkylene-O—C₁₋₆alkyl-OC(O)R¹, C₁₋₆alkylene-O—C₁₋₆haloalkyl-OC(O)R¹, C₂₋₆alkenylene-O—C₁₋₆haloalkyl-O—C(O)R¹, C₂₋₆alkenylene-O—C₁₋₆haloalkyl-O—C(O)R¹, C₁₋₆alkylene-C₃₋₁₀cycloalkyl-O—C(O)R¹, C₁₋₆alkylene-heterocycloalkyl-O—C(O)R¹, C₁₋₆alkylene-aryl-O—C(O)R¹, C₁₋₆alkylene-heteroaryl-O—C(O)R¹, C₁₋₁₀alkyl-C(O)OR¹, C₂₋₁₀alkenyl-C(O)OR¹, C₂₋₁₀alkynyl-C(O)OR¹, C₁₋₁₀haloalkyl-C(O)OR¹, C₁₋₁₀cyanoalkyl-C(O)OR¹, C₁₋₁₀alkoxy-C(O)OR¹, C₂₋₁₀alkenyloxy-C(O)OR¹, C₃₋₁₀cycloalkyl-C(O)OR¹, heterocycloalkyl-C(O)OR¹, aryl-C(O)OR¹, heteroaryl-C(O)OR¹, C₁₋₆alkylene-O—C₁₋₆alkyl-C(O)OR¹, C₁₋₆alkylene-O—C₁₋₆haloalkyl-C(O)OR¹, C₂₋₆alkenylene-O—C₁₋₆haloalkyl-C(O)OR¹, C₂₋₆alkenylene-O—C₁₋₆haloalkyl-C(O)OR¹, C₁₋₆alkylene-C₃₋₈cycloalkyl-C(O)OR¹, C₁₋₆alkylene-heterocycloalkyl-C(O)OR¹, C₁₋₆alkylene-aryl-C(O)OR¹, C₁₋₆alkylene-heteroaryl-C(O)OR¹, C₁₋₆alkylene-O—R¹, C₁₋₆alkylene-C(O)R¹, C₁₋₆alkylene-O—C(O)R¹, C₁₋₆alkylene-C(O)OR¹, C₁₋₆alkylene-O—C(O)OR¹, C₁₋₆alkylene-NR¹R¹, C₁₋₆alkylene-C(O)NR¹R², C₁₋₆alkylene-NR¹C(O)R², C₁₋₆alkylene-NR¹C(O)NR³R², C₁₋₆alkylene-S—R¹, C₁₋₆alkylene-S(O)R¹, C₁₋₆alkylene-SO₂R¹, C₁₋₆alkylene-SO₂NR¹R², C₁₋₆alkylene-NR¹SO₂R², C₁₋₆alkylene-NR³SO₂NR¹R², C(O)NR¹R² and C₁₋₆alkylene-NR¹C(O)OR², wherein R is optionally substituted with C₁₋₄alkyl and any cyclic or heterocyclic moiety is optionally fused to a further cyclic or heterocyclic moiety;
R¹, R² and R³ are each independently selected from the group consisting of H, C₁₋₆alkyl, C₁₋₆haloalkyl, C₂₋₆alkenyl, C₂₋₆alkynyl, C₃₋₁₀cycloalkyl, C₁₋₆alkylene-C₃₋₁₀cycloalkyl, heterocycloalkyl, aryl, C₁₋₆alkylene-aryl, C₁₋₆alkylene-heterocycloalkyl, heteroaryl, and C₁₋₆alkylene-heteroaryl, wherein any cyclic or heterocyclic moiety is optionally fused to a further cyclic or heterocyclic moiety.

According to an aspect of the application there is provided a process for the synthesis of difluoromethyl ethers of Formula (I) comprising the steps of: (1) thioformylation phenolic or aliphatic hydroxyl groups (alcohols or phenols) with Vilsmeier reagent [commercially available or generated in situ from Dimethlformamide (DMF) and oxalyl chloride] followed by Hydrogen sulfide (H₂S) in presence of pyridine (Scheme 3) (Heterocycles 1989, 28(2), 887-98] or Sodium Hydrosulfide, Monohydrate (Synlett 2009, 3139-3142) (Scheme 3).

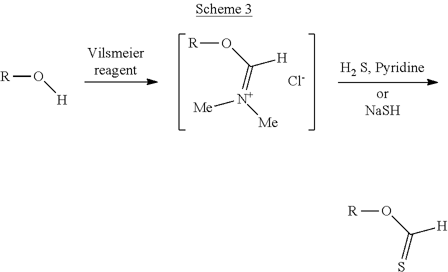

(2) converting thioformyl esters into the corresponding difluoromethyl ethers with 2,2-difluoro-1,3-dimethylimidazolidine generated in situ from 2-chloro-1,3-dimethyl-4,5-dihydroimidazol-1-ium chloride and potassium fluoride in acetonitrile (Scheme 4)

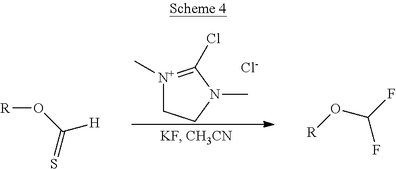

The present application also includes a composition comprising one or more difluoromethyl ether compounds of the application and a carrier. In an embodiment, the composition is a pharmaceutical composition or a precursor for a pharmaceutical composition comprising one or more compounds of the application and a pharmaceutically acceptable carrier.

In a further embodiment, the difluoromethyl ether compounds of the application are used as procursors for medicaments. Accordingly, the application also includes a difluoromethyl-ether compound of the application for use as a medicament.

The application additionally provides a process for the preparation of compounds of Formula (I). General and specific processes are discussed in more detail and set forth in the Examples below.

In an embodiment, the present process utilizes safer reaction conditions for difluoromethylation.

In another embodiment, the present process is more effective and efficient one pot route for the synthesis of difluoromethyl ether compounds using environment friendly and readily accessible reagents and operational simplicity for commercial scale up.

In a further embodiment, the newly developed process produces difluoromethyl ether compounds at a lower cost and high purity.

The following example further illustrates certain specific aspects and embodiments of the application in detail and is not intended to limit the scope of the application.

The introduction of a halogen atom into a molecule also provides the opportunity for the use of the molecule in radiolabeling applications. For example, ¹⁸F is used as a radiolabel tracer in the sensitive technique of Positron Emission Tomography (PET). Accordingly, the present application also includes methods of using the compounds of the application for diagnostic and/or imaging purposes;

Other features and advantages of the present application will become apparent from the following detailed description. It should be understood, however, that the detailed description and the specific examples, while indicating embodiments of the application, are given by way of illustration only and the scope of the claims should not be limited by these embodiments, but should be given the broadest interpretation consistent with the description as a whole.

### DETAILED DESCRIPTION

### I. Definitions

Unless otherwise indicated, the definitions and embodiments described in this and other sections are intended to be applicable to all embodiments and aspects of the application herein described for which they are suitable as would be understood by a person skilled in the art. Unless otherwise specified within this application or unless a person skilled in the art would understand otherwise, the nomenclature used in this application generally follows the examples and rules stated in “Nomenclature of Organic Chemistry” (Pergamon Press, 1979), Sections A, B, C, D, E, F, and H. Optionally, a name of a compound may be generated using a chemical naming program: ACD/ChemSketch, Version 5.09/September 2001, Advanced Chemistry Development, Inc., Toronto, Canada.

The term “compound of the application” or “compound of the present application” and the like as used herein refers to a compound of Formula I, and pharmaceutically acceptable salts, solvates and/or prodrugs thereof.

The term “difluoromethyl ether compounds of the application” as used herein refers to difluoromethyl ether compounds prepared using the methods disclosed herein.

The term “and/or” as used herein means that the listed items are present, or used, individually or in combination. In effect, this term means that “at least one of” or “one or more” of the listed items is used or present. The term “and/or” with respect to pharmaceutically acceptable salts, solvates and/or prodrugs thereof means that the compounds of the application exist as individual salts, hydrates or prodrugs, as well as a combination of, for example, a salt of a solvate of a compound of the application or a salt of a prodrug of a compound of a compound of the application.

As used in the present application, the singular forms “a”, “an” and “the” include plural references unless the content clearly dictates otherwise. For example, an embodiment including “a compound” should be understood to present certain aspects with one compound, or two or more additional compounds.

In embodiments comprising an “additional” or “second” component, such as an additional or second compound, the second component as used herein is chemically different from the other components or first component. A “third” component is different from the other, first, and second components, and further enumerated or “additional” components are similarly different.

In understanding the scope of the present application, the term “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open ended and do not exclude additional, unrecited elements or process steps.

The term “consisting” and its derivatives, as used herein, are intended to be closed terms that specify the presence of the stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The term “suitable” as used herein means that the selection of the particular compound or conditions would depend on the specific synthetic manipulation to be performed, and the identity of the molecule(s) to be transformed and/or the specific use for the compound, but the selection would be well within the skill of a person trained in the art. In embodiments of the present application, the difluoromethyl ether compounds described herein may have at least one asymmetric center. Where compounds possess more than one asymmetric center, they may exist as diastereomers. It is to be understood that all such isomers and mixtures thereof in any proportion are encompassed within the scope of the present application. It is to be further understood that while the stereochemistry of the compounds may be as shown in any given compound listed herein, such compounds may also contain certain amounts (for example, less than 20%, suitably less than 10%, more suitably less than 5%) of difluoromethyl ether compounds of the present application having alternate stereochemistry. It is intended that any optical isomers, as separated, pure or partially purified optical isomers or racemic mixtures thereof are included within the scope of the present application.

In embodiments of the present application, the difluoromethyl ether compounds described herein having a double bond can exist as geometric isomers, for example cis or trans isomers. It is to be understood that all such geometric isomers and mixtures thereof in any proportion are encompassed within the scope of the present application. It is to be further understood that while the stereochemistry of these difluoromethyl ether compounds may be as shown in any given compound listed herein, such compounds may also contain certain amounts (for example, less than 20%, suitably less than 10%, more suitably less than 5%) of difluoromethyl ether compounds of the present application having alternate stereochemistry.

The difluoromethyl ether compounds of the present application may also exist in different tautomeric forms and it is intended that any tautomeric forms which the compounds form, as well as mixtures thereof, are included within the scope of the present application.

The difluoromethyl ether compounds of the present application may further exist in varying polymorphic forms and it is contemplated that any polymorphs, or mixtures thereof, which form are included within the scope of the present application.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies or unless the context suggests otherwise to a person skilled in the art.

The expression “proceed to a sufficient extent” as used herein with reference to the reactions or method steps disclosed herein means that the reactions or process steps proceed to an extent that conversion of the starting material or substrate to product is maximized. Conversion may be maximized when greater than about 5, 10, 15, 20, 25, 30, 35, 40, 45, 50, 55, 60, 65, 70, 75, 80, 85, 90, 95 or 100% of the starting material or substrate is converted to product.

The term “organic compound” as used herein means any chemical compound comprising carbon and hydrogen atoms, and optionally one or more heteroatoms, such as, but not limited to P, N, O and/or S and that is compatible with the reaction conditions used in the processes of the application. The identification and/or selection of organic compounds that are compatible with the reaction conditions used in the processes of the application can be made by a person skilled in the art.

The term “compatible with” as used herein means that a compound will not degrade to an appreciable extent and/or that unwanted side reactions will not occur to an appreciable extent when that compound is subjected to the reaction conditions used in the processes of the application.

The term “appreciable extent” as used herein means an amount that, when considering all of the factors in the preparation of a compound, the amount of degradation and/or side reactions does not make the process commercially undesirable. For example, the amount of degration and/or side reactions is less than about 50%, 45%, 40%, 35%, 30%, 25%, 20%, 15%, 10% or 5%.

The term “Vilsmeier reagent” as used herein refers to the reagent formed from the reaction of dimethyl formamide (DMF) and a chlorinating reagent, such as oxalyl chloride.

The term “sulfurating reagent” as used herein refers to any reagent that that will incorporate sulfur into the intermediate formed by the reaction of the Vilsmeier reagent with the alcohol to form the thioformylester.

The term “seven-membered” or “7-membered” as used herein as a prefix refers to a group having a ring that contains seven ring atoms.

The term “six-membered” or “6-membered” as used herein as a prefix refers to a group having a ring that contains six ring atoms.

The term “five-membered” or “5-membered” as used herein as a prefix refers to a group having a ring that contains five ring atoms.

The term “hydrocarbon” as used herein, whether it is used alone or as part of another group, refers to any structure comprising only carbon and hydrogen atoms up to 14 carbon atoms.

The term “hydrocarbon radical” or “hydrocarbyl” as used herein, whether it is used alone or as part of another group, refers to any structure derived as a result of removing a hydrogen atom from a hydrocarbon.

The term “hydrocarbylene” as used herein, whether it is used alone or as part of another group, refers to any structure derived as a result of removing a hydrogen atom from two ends of a hydrocarbon.

The term “alkyl” as used herein, whether it is used alone or as part of another group, means straight or branched chain, saturated alkyl groups. The number of carbon atoms that are possible in the referenced alkyl group are indicated by the prefix “Cₙ₁₋ₙ₂”. For example, the term C₁₋₁₀alkyl means an alkyl group having 1, 2, 3, 4, 5, 6, 7, 8, 9 or 10 carbon atoms.

The term “alkylene” as used herein, whether it is used alone or as part of another group, means straight or branched chain, saturated alkylene group; that is, a saturated carbon chain that contains substituents on two of its ends. The number of carbon atoms that are possible in the referenced alkylene group are indicated by the prefix “Cₙ₁₋ₙ₂”. For example, the term C₁₋₁₀alkylene means an alkylene group having 1, 2, 3, 4, 5, 6, 7, 8, 9 or 10 carbon atoms.

The term “alkenyl” as used herein, whether it is used alone or as part of another group, means straight or branched chain, unsaturated alkyl groups containing at least one double bond. The number of carbon atoms that are possible in the referenced alkenyl group are indicated by the prefix “Cₙ₁₋ₙ₂”. For example, the term C₂₋₁₀alkenyl means an alkenyl group having 2, 3, 4, 5, 6, 7, 8, 9 or 10 carbon atoms and at least one double bond.

The term “alkenylene” as used herein means straight or branched chain, unsaturated alkenylene group, that is, an unsaturated carbon chain that contains substituents on two of its ends. The number of carbon atoms that are possible in the referenced alkylene group are indicated by the prefix “Cₙ₁₋ₙ₂”. For example, the term C₂₋₁₀alkenylene means an alkenylene group having 2, 3, 4, 5, 6, 7, 8, 9 or 10 carbon atoms and at least 1, for example 1-3, 1-2 or 1 double bond.

The term “alkynyl” as used herein, whether it is used alone or as part of another group, means straight or branched chain unsaturated alkyl groups containing at least one triple bond. The number of carbon atoms that are possible in the referenced alkynyl group are indicated by the prefix “Cₙ₁₋ₙ₂”. For example, the term C₂₋₆alkynyl means an alkynyl group having 2, 3, 4, 5 or 6 carbon atoms and at least one triple bond.

The term “alkynylene” as used herein means straight or branched chain, unsaturated alkynylene group, that is, an unsaturated carbon chain that contains substituents on two of its ends. The number of carbon atoms that are possible in the referenced alkylylene group are indicated by the prefix “Cₙ₁₋ₙ₂”. For example, the term C₂₋₆alkynylene means an alkynylene group having 2, 3, 4, 5 or 6 carbon atoms and at least 1 triple bond. The term “haloalkyl” or “alkylhalo” as used herein refers to an alkyl group wherein one or more, including all of the hydrogen atoms are replaced by a halogen atom. In an embodiment, the halogen is fluorine, in which case the haloalkyl is referred to herein as a “fluoroalkyl” group or an “alkylfluoro” group. In another embodiment, the haloalkyl or alkylhalo comprises at least one —CHF₂ group.

The term “haloalkylene” as used herein refers to an alkylene group wherein one or more, including all of the hydrogen atoms are replaced by a halogen atom. In an embodiment, the halogen is fluorine, in which case the haloalkylene is referred to herein as a “fluoroalkylene” group. In another embodiment, the haloalkylene comprises a branched fluoroalkylene having at least one O—CHF₂ group.

The term “cyanoalkyl” or “alkylcyano” and the like as used herein refers to an alkyl group that is substituted by at least one cyano group. The number of carbon atoms that are possible in the referenced cyanoalkyl group are indicated by the prefix “Cₙ₁₋ₙ₂”. For example, the term C₁₋₁₀cyanoalkyl means an alkyl group having 1, 2, 3, 4, 5, 6, 7, 8, 9 or 10 carbon atoms and at least one cyano group attached thereto.

The term “alkoxy” as used herein, whether it is used alone or as part of another group, refers to the group “alkyl-O-” or “—O-alkyl”. The number of carbon atoms that are possible in the referenced alkoxy group are indicated by the prefix “Cₙ₁₋ₙ₂”. For example, the term C₁₋₁₀alkoxy means an alkyl group having 1, 2, 3, 4, 5, 6, 7, 8, 9 or 10 carbon atoms bonded to the oxygen atom. Exemplary alkoxy groups include without limitation methoxy, ethoxy, propoxy, isopropoxy, butoxy, t-butoxy and isobutoxy.

The term “cycloalkyloxy” as used herein, whether it is used alone or as part of another group, refers to the group “cycloalkyl-O”. The number of carbon atoms that are possible in the referenced cycloalkyloxy group are indicated by the prefix “Cₙ₁₋ₙ₂”. For example, the term C₃₋₈cycloalkoxy means a cycloalkyl group having 3, 4, 5, 6, 7 or 8 carbon atoms bonded to the oxygen atom.

The term “alkenyloxy” as used herein, whether it is used alone or as part of another group, refers to the group “alkenyl-O-”. The number of carbon atoms that are possible in the referenced alkenyloxy group are indicated by the prefix “Cₙ₁₋ₙ₂”. For example, the term C₂₋₁₀alkenyloxy means an alkenyl group having 2, 3, 4, 5, 6, 7, 8, 9 or 10 carbon atoms and at least one double bond bonded to the oxygen atom. An exemplary alkenyloxy group is an allyloxy group.

The term “alkynyloxy” as used herein, whether it is used alone or as part of another group, refers to the group “alkynyl-O-”. The number of carbon atoms that are possible in the referenced alkynyloxy group are indicated by the prefix “Cₙ₁₋ₙ₂”. For example, the term C₂₋₁₀alkynyloxy means an alkynyl group having 2, 3, 4, 5, 6, 7, 8, 9 or 10 carbon atoms and at least one triple bond bonded to the oxygen atom. An exemplary alkynyloxy group is a propargyloxy group.

The term “aryloxy” as used herein, whether it is used alone or as part of another group, refers to the group “aryl-O-”. The number of carbon atoms that are possible in the referenced aryloxy group are indicated by the prefix “Cₙ₁₋ₙ₂”. In an embodiment of the present disclosure, the aryl group contains 6, 9, 10 or 14 atoms such as phenyl, naphthyl, indanyl or anthracenyl.

The term “cycloalkyl” as used herein, whether it is used alone or as part of another group, means a saturated carbocylic group containing a number of carbon atoms and one or more rings. The number of carbon atoms that are possible in the referenced cycloalkyl group are indicated by the numerical prefix “Cₙ₁₋ₙ₂”. For example, the term C₃₋₁₀cycloalkyl means a cycloalkyl group having 3, 4, 5, 6, 7, 8, 9 or 10 carbon atoms.

The term “cycloalkylene” as used herein refers to a cycloalkyl group that contains substituents on two of its ends.

The term “aryl” as used herein, whether it is used alone or as part of another group, refers to cyclic groups containing 6 to 20 carbon atoms that contain at least one aromatic ring. In an embodiment of the application, the aryl group contains from 6, 9 or 10, such as phenyl, naphthyl or indanyl.

The term “arylene” as used herein refers to an aryl group that contains substituents on two of its ends.

The term “heteroarylene” as used herein refers to a heteroaryl group that contains substituents on two of its ends.

The term “heterocycloalkyl” as used herein, whether it is used alone or as part of another group, refers to cyclic groups containing 3 to 10 atoms, suitably 3 to 6 atoms, and at least one non-aromatic ring in which one or more of the atoms are a heteromoiety selected from N, NH, O, NC₁₋₆alkyl and S. Heterocycloalkyl groups are either saturated or unsaturated (i.e. contain one or more double bonds) and contain one or more than one ring (i.e. are polycyclic). When a heterocycloalkyl group contains more than one ring, the rings may be fused, bridged, spirofused or linked by a bond. When a heterocycloalkyl group contains the prefix Cₙ₁₋ₙ₂ this prefix indicates the number of carbon atoms in the corresponding carbocyclic group, in which one or more, suitably 1 to 5, of the ring atoms is replaced with a heteromoiety as defined above.

A first ring group being “fused” with a second ring group means the first ring and the second ring share at least two adjacent atoms there between.

A first ring group being “bridged” with a second ring group means the first ring and the second ring share at least two non-adjacent atoms there between.

A first ring group being “spirofused” with a second ring group means the first ring and the second ring share one atom there between.

Heterocycloalkyl includes monocyclic heterocycloalkyls such as but not limited to aziridinyl, oxiranyl, thiiranyl, azetidinyl, oxetanyl, thietanyl, pyrrolidinyl, pyrrolinyl, imidazolidinyl, pyrazolidinyl, pyrazolinyl, dioxolanyl, sulfolanyl, 2,3-dihydrofuranyl, 2,5-dihydrofuranyl, tetrahydrofuranyl, thiophanyl, piperidinyl, 1,2,3,6-tetrahydropyridinyl, piperazinyl, morpholinyl, thiomorpholinyl, pyranyl, thiopyranyl, 2,3-dihydropyranyl, tetrahydropyranyl, 1,4-dihydropyridinyl, 1,4-dioxanyl, 1,3-dioxanyl, dioxanyl, homopiperidinyl, 2,3,4,7-tetrahydro-1H-azepinyl, homopiperazinyl, 1,3-dioxepanyl, 4,7-dihydro-1,3-dioxepinyl, and hexamethylene oxidyl. Additionally, heterocycloalkyl includes polycyclic heterocycloalkyls such as but not limited to pyrolizidinyl and quinolizidinyl. In addition to the polycyclic heterocycloalkyls described above, heterocycloalkyl includes polycyclic heterocycloalkyls wherein the ring fusion between two or more rings includes more than one bond common to both rings and more than two atoms common to both rings. Examples of such bridged heterocycles include but are not limited to quinuclidinyl, diazabicyclo[2.2.1]heptyl and 7-oxabicyclo[2.2.1]heptyl.

The term “heteroaryl” as used herein refers to cyclic groups containing from 5 to 20 atoms, suitably 5 to 10 atoms, at least one aromatic ring and at least one a heteromoiety selected from O, S, N, NH and NC₁₋₆alkyl. Heteroaryl groups contain one or more than one ring (i.e. are polycyclic). When a heteroaryl group contains more than one ring, the rings may be fused, bridged, spirofused or linked by a bond. When a heteroaryl group contains the prefix Cₙ₁₋ₙ₂ this prefix indicates the number of carbon atoms in the corresponding carbocyclic group, in which one or more, suitably 1 to 5, of the ring atoms is replaced with a heteromoiety as defined above.

Heteroaryl includes for example, pyridinyl, pyrazinyl, pyrimidinyl, triazinyl, pyridazinyl. thienyl, furyl, furazanyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

Heteroaryl also includes polycyclic heteroaryls such as but not limited to indolyl, indolinyl, isoindolinyl, quinolinyl, tetrahydroquinolinyl, isoquinolinyl, tetrahydroisoquinolinyl, 1,4-benzodioxanyl, coumarinyl, dihydrocoumarinyl, benzofuranyl, 2,3-dihydrobenzofuranyl, isobenzofuranyl, chromenyl, chromanyl, isochromanyl, xanthenyl, phenoxathiinyl, thianthrenyl, indolizinyl, isoindolyl, indazolyl, purinyl, phthalazinyl, naphthyridinyl, quinoxalinyl, quinazolinyl, cinnolinyl, pteridinyl, phenanthridinyl, perimidinyl, phenanthrolinyl, phenazinyl, phenothiazinyl, phenoxazinyl, 1,2-benzisoxazolyl, benzothiophenyl, benzoxazolyl, benzthiazolyl, benzimidazolyl, benztriazolyl, thioxanthinyl, carbazolyl, carbolinyl and acridinyl.

A five-membered heteroaryl is a heteroaryl with a ring having five ring atoms, where 1, 2 or 3 ring atoms are a heteromoiety selected from O, S, NH and NC₁₋₆alkyl. Exemplary five-membered heteroaryls include but are not limited to thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl.

A six-membered heteroaryl is a heteroaryl with a ring having six ring atoms wherein 1, 2 or 3 ring atoms are a heteromoiety selected from O, S, NH and NC₁₋₆alkyl. Exemplary six-membered heteroaryls include but are not limited to pyridinyl, pyrazinyl, pyrimidinyl, triazinyl and pyridazinyl.

The term “cyclic moiety” as used herein refers to any cycloalkyl, aryl, heteroaryl or heterocycloalkyl group as defined herein.

The term “heteromoiety” as used herein refers to a group of atoms containing at least one heteroatom.

As a prefix, the term “substituted” as used herein refers to a structure, molecule or group in which one or more available hydrogen atoms are replaced with one or more other chemical groups. In an embodiment, the chemical group is a C₁₋₄alkyl. In another embodiment, the chemical group is a C₁₋₁₂alkyl or a chemical group that contains one or more heteroatoms selected from N, O, S, F, Cl, Br, I and P. Exemplary chemical groups containing one or more heteroatoms include heterocycloalkyl, heteroaryl, —NO₂, —OR, —R′OR, —Cl, —Br, —I, —F, —CF₃, —C(O)R, —NR₂, —SR, —SO₂R, —S(O)R, —CN, —C(O)OR, —C(O)NR₂, —NRC(O)R, —NRC(O)OR, —R′NR₂, oxo (═O), imino (═NR), thio (═S), and oximino (═N—OR), wherein each “R” is hydrogen or a C₁₋₁₂alkyl and “R′” is a C₁₋₁₂alkylene. For example, substituted phenyl may refer to nitrophenyl, pyridylphenyl, methoxyphenyl, chlorophenyl, aminophenyl, etc., wherein the nitro, pyridyl, methoxy, chloro, and amino groups may replace any available hydrogen on the phenyl ring.

As a suffix, the term “substituted” as used herein in relation to a first structure, molecule or group, followed by one or more variables or names of chemical groups, refers to a second structure, molecule or group that results from replacing one or more available hydrogen atoms of the first structure, molecule or group with the one or more variables or named chemical groups. For example, a “phenyl substituted by nitro” refers to nitrophenyl.

The term “available”, as in “available hydrogen atoms” or “available atoms” refers to atoms that would be known to a person skilled in the art to be capable of replacement by a substituent.

The term “optionally substituted” refers to groups, structures, or molecules that are either unsubstituted or are substituted with one or more substituents.

The term “amine” or “amino” as used herein, whether it is used alone or as part of another group, refers to radicals of the general formula —NRR′, wherein R and R′ are each independently selected from hydrogen or an alkyl group, for example C₁₋₆alkyl.

The term “halo” or “halogen” as used herein, whether it is used alone or as part of another group, refers to a halogen atom and includes fluoro, chloro, bromo and iodo.

The term “acac” as used herein refers to acetylacetonate.

The terms “Boc” and “t-Boc” and the like as used herein refer to the group tert-butoxycarbonyl.

DCM as used herein refers to dichloromethane.

DIPEA as used herein refers to N,N-diisopropyl ethylamine.

DMF as used herein refers to dimethylformamide.

DMSO as used herein refers to dimethylsulfoxide.

Et₂O as used herein refers to diethylether.

EtOAc as used herein refers to ethyl acetate.

Et as used herein refers to the group ethyl.

Fmoc as used herein refers to the group 9-fluorenylmethyloxycarbonyl.

The term “hr(s)” as used herein refers to hour(s).

The term “min(s)” as used herein refers to minute(s).

HOBt as used herein refers to N-hydroxybenzotriazole.

HBTU as used herein refers to O-(benzotriazol-1-yl)-N,N,N′,N′-tetramethyluronium hexafluorophosphate.

MeOH as used herein refers to methanol.

Me as used herein refers to the group methyl.

t-BuLi as used herein refers to tert-butyllithium.

ON as used herein refers to overnight.

RT as used herein refers to room temperature.

TEA as used herein refers to triethylamine.

TFA as used herein refers to trifluoroacetic acid.

THF as used herein refers to tetrahydrofuran.

t-Bu as used herein refers to the group tertiary butyl.

SPE as used herein refers to solid phase extraction, for example using columns containing silica gel for mini-chromatography.

The term “sat.” as used herein refers to saturated.

The term “protecting group” or “PG” and the like as used herein refers to a chemical moiety which protects or masks a reactive portion of a molecule to prevent side reactions in those reactive portions of the molecule, while manipulating or reacting a different portion of the molecule. After the manipulation or reaction is complete, the protecting group is removed under conditions that do not degrade or decompose the remaining portions of the molecule. The selection of a suitable protecting group can be made by a person skilled in the art. Many conventional protecting groups are known in the art, for example as described in “Protective Groups in Organic Chemistry” McOmie, J. F. W. Ed., Plenum Press, 1973, in Greene, T. W. and Wuts, P. G. M., “Protective Groups in Organic Synthesis”, John Wiley & Sons, 3^{rd} Edition, 1999 and in Kocienski, P. Protecting Groups, 3rd Edition, 2003, Georg Thieme Verlag (The Americas). Examples of suitable protecting groups include, but are not limited to t-Boc, cbz, Ac, Ts, Ms, silyl ethers such as TMSi, TBDMS, TBDPS, Tf, Ns, Bn, Fmoc, benzoyl, dimethoxytrityl, methoxyethoxymethyl ether, methoxymethyl ether, pivaloyl, p-methyoxybenzyl ether, tetrahydropyranyl, trityl, ethoxyethyl ethers, carbobenzyloxy, benzoyl and the like.

Cbz as used herein refers to the group carboxybenzyl.

Ac as used herein refers to the group acetyl.

Ts (tosyl) as used herein refers to the group p-toluenesulfonyl.

Ms as used herein refers to the group methanesulfonyl.

TMS as used herein refers to tetramethylsilane.

TMSi as used herein refers to the group trimethylsilyl.

TBDMS as used herein refers to the group t-butyldimethylsilyl.

TBDPS as used herein refers to the group t-butyldiphenylsilyl.

Tf as used herein refers to the group trifluoromethanesulfonyl.

Ns as used herein refers to the group naphthalene sulphonyl.

Bn as used herein refers to the group benzyl.

The term “cell” as used herein refers to a single cell or a plurality of cells and includes a cell either in a cell culture or in a subject.

The term “subject” as used herein includes all members of the animal kingdom including mammals, and suitably refers to humans. Thus the methods and uses of the present application are applicable to both human therapy and veterinary applications. In an embodiment of the present application, the subject is a mammal. In another embodiment, the subject is human.

The term “pharmaceutically acceptable” means compatible with the treatment of subjects, for example humans.

The term “pharmaceutically acceptable carrier” means a non-toxic solvent, dispersant, excipient, adjuvant or other material which is mixed with the active ingredient in order to permit the formation of a pharmaceutical composition; i.e., a dosage form capable of administration to a subject.

The term “pharmaceutically acceptable salt” means either an acid addition salt or a base addition salt which is suitable for, or compatible with the treatment of subjects.

An acid addition salt suitable for, or compatible with, the treatment of subjects is any non-toxic organic or inorganic acid addition salt of any basic compound. Basic compounds that form an acid addition salt include, for example, compounds comprising an amine group. Illustrative inorganic acids which form suitable salts include hydrochloric, hydrobromic, sulfuric, nitric and phosphoric acids, as well as acidic metal salts such as sodium monohydrogen orthophosphate and potassium hydrogen sulfate. Illustrative organic acids which form suitable salts include mono-, di- and tricarboxylic acids. Illustrative of such organic acids are, for example, acetic, trifluoroacetic, propionic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, mandelic, salicylic, 2-phenoxybenzoic, p-toluenesulfonic acid and other sulfonic acids such as methanesulfonic acid, ethanesulfonic acid and 2-hydroxyethanesulfonic acid. Either the mono- or di-acid salts can be formed, and such salts can exist in either a hydrated, solvated or substantially anhydrous form. In general, acid addition salts are more soluble in water and various hydrophilic organic solvents, and generally demonstrate higher melting points in comparison to their free base forms. The selection criteria for the appropriate salt will be known to one skilled in the art. Other non-pharmaceutically acceptable salts such as but not limited to oxalates may be used, for example in the isolation of compounds of the application for laboratory use, or for subsequent conversion to a pharmaceutically acceptable acid addition salt.

In another embodiment of the present application, the difluoromethyl ether compounds of Formula I is converted to a pharmaceutically acceptable salt or solvate thereof, in particular an acid addition salt such as a hydrochloride, hydrobromide, phosphate, acetate, fumarate, maleate, tartrate, citrate, methanesulphonate or p-toluenesulphonate.

A base addition salt suitable for, or compatible with, the treatment of subjects is any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide as well as ammonia. Illustrative organic bases which form suitable salts include aliphatic, alicyclic or aromatic organic amines such as isopropylamine, methylamine, trimethylamine, picoline, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. [See, for example, S. M. Berge, et al., “Pharmaceutical Salts,” J. Pharm. Sci. 1977, 66, 1-19]. The selection of the appropriate salt may be useful so that an ester functionality, if any, elsewhere in a compound is not hydrolyzed. The selection criteria for the appropriate salt will be known to one skilled in the art.

In general, prodrugs will be functional derivatives of the compounds of the application which are readily convertible in vivo into the compound from which it is notionally derived. In an embodiment, produgs of the compounds of the application are conventional esters formed with available hydroxy, thiol, amino or carboxyl groups. For example, an available OH and/or NH₂ in the compounds of the application is acylated using an activated acid in the presence of a base, and optionally, in inert solvent (e.g. an acid chloride in pyridine). Some common esters which have been utilized as prodrugs are phenyl esters, aliphatic (C₁-C₂₄) esters, acyloxymethyl esters, carbamates and amino acid esters. In certain instances, the prodrugs of the compounds of the application are those in which the hydroxy and/or amino groups in the compounds are masked as groups which can be converted to hydroxy and/or amino groups in vivo. Conventional procedures for the selection and preparation of suitable prodrugs are described, for example, in “Design of Prodrugs” ed. H. Bundgaard, Elsevier, 1985.

The term “solvate” as used herein means a compound, or a salt or prodrug of a compound, wherein molecules of a suitable solvent are incorporated in the crystal lattice. A suitable solvent is physiologically tolerable at the dosage administered. Examples of suitable solvents are ethanol, water and the like. When water is the solvent, the compound is referred to as a “hydrate”. The formation of solvates of the compounds of the application will vary depending on the compound and the solvate. In general, solvates are formed by dissolving the compound in the appropriate solvent and isolating the solvate by cooling or using an antisolvent. The solvate is typically dried or azeotroped under ambient conditions. The selection of suitable conditions to form a particular solvate can be made by a person skilled in the art.

### II. Processes of the Application

The present application includes a process for the preparation of difluoromethyl ethers the process comprising:
a) reacting a suitable alcohol with Vilsmeier reagent followed by a sulfurating reagent under conditions to provide a thioformyl ester; and
b) reacting the thioformyl ester of step (a) with 2,2-difluoro-1,3-dimethylimidazolidine under conditions to provide the difluoromethyl ether.

In an embodiment the suitable alcohol is any suitable organic compound comprising an alcohol or hydroxyl (“OH”) group.

In a further embodiment, the suitable organic alcohol is any alcohol that is compatible with the Vilsmeier reagent.

In an embodiment of the application, the conditions to provide a thioformyl ester comprise reacting any suitable alcohol with Vilsmeier reagent. In an embodiment, the Vilsmeier reagent is generated in situ in inert solvents at temperature and time sufficient for the conversion to proceed to a sufficient extent. The Vilsmeier reagent is the reaction product of a substituted amide with an oxychloride to provide a substituted chloroimminium ion. In an embodiment of the present application, the Vilsmeier reagent is generated in situ from DMF and oxalyl chloride. Examples of non-limiting reaction temperatures include, but are not limited to, −20° C. to about 10° C. or −5° C. to about 5° C. Examples of non-limiting reaction times are about 5 minutes to about 1 hour or about 15 minutes to about 30 minutes. Examples of non-limiting inert solvents include, but are not limited to halogenated solvents. In an embodiment, the halogenated solvent is dichloromethane.

In an embodiment, the conditions to provide the thioformyl ester further comprises the addition of a suitable organic alcohol neat or in combination with an inert solvent to a reaction mixture comprising the in situ generated Vilsmeier reagent. Following the addition, a sufurating reagent, such as hydrogen sulfide (H₂S) in the presence of pyridine or an equivalent suitable salt of hydrogen sulfide (such as NaSH) is added to the reaction mixture in an inert solvent at temperature and time sufficient to proceed to a sufficient extent. In an embodiment, the solution comprising the suitable salt of hydrogen sulfide is added to the reaction mixture quickly, with vigorous stirring to allow the conversion to proceed to a sufficient extent. In another embodiment, the solution comprising the suitable salt of hydrogen sulfide is as concentrated as possible. Examples of non-limiting reaction temperatures include, but are not limited to, −40° C. to about 10° C., −30° C. to about 5° C. or −20° C. to about −10° C. Examples of non-limiting reaction times include, but are not limited to 5 minutes to about 1 hour or about 15 minutes to about 30 minutes. Examples of non-limiting inert solvents include organic solvents and aqueous solvents. In an embodiment, the inert solvent is an organic solvent. In a further embodiment, the organic solvent is acetonitrile. In an embodiment, the inert solvent used for the suitable salt of hydrogen sulfide is an aqueous solvent. In a further embodiment, the aqueous solvent is water.

In an embodiment, the conditions to provide the difluoromethylether of Formula (I) comprises reacting the thioformyl ester with 2,2-difluoro-1,3-dimethylimidazoline in inert solvents at temperatures and times sufficient for the conversion to proceed to a sufficient extent. Examples of non-limiting temperatures include, but are not limited to, −10° C. to about 100° C., −5° C. to about 50° C. or 0° C. to about 30° C. Examples of non-limiting reaction times include, but are not limited to 5 minutes to about 10 hours, 15 minutes to about 5 hours or about 30 minutes to about 3 hours. Examples of non-limiting inert solvents include but are not limited to organic solvents. In an embodiment, the inert solvent is acetonitrile.

In an embodiment, 2,2-difluoro-1,3-dimethylimidazoline is generated in situ from 2-chloro-1,3-dimethyl-4,5-dihydroimidazol-1-ium chloride and potassium fluoride in an inert solvent at temperature and time sufficient for the conversion to proceed to a sufficient extent. Examples of non-limiting temperatures include, but are not limited to, 10° C. to about 120° C., 50° C. to about 100° C. or 70° C. to about 90° C. Examples of non-limiting reaction times include, but are not limited to 5 hours to about 30 hours or about 10 hours to about 20 hours. Examples of non-limiting inert solvents include but are not limited to organic solvents. In an embodiment, the inert solvent is acetonitrile.

In another aspect, the present application further includes a process for the preparation of difluoromethyl ethers of Formula (I) or pharmaceutically acceptable salts, solvates and/or prodrug thereof:

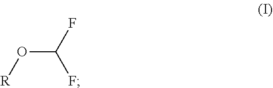

the process comprising:
a) reacting a compound of Formula (II) with Vilsmeier reagent followed by a sulfurating reagent under conditions to provide the compound of Formula (III):

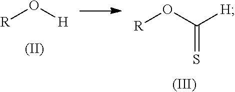

b) reacting a compound of Formula (III) with 2,2-difluoro-1,3-dimethylimidazolidine under conditions to provide the compound of Formula (I):

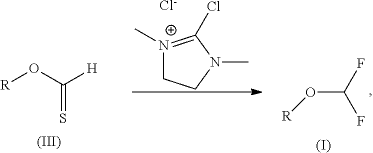

wherein
R is selected from D/L-amino acids, C₁₋₁₀alkyl, C₂₋₁₀alkenyl, C₂₋₁₀alkynyl, C₁₋₁₀haloalkyl, C₁₋₁₀cyanoalkyl, C₁₋₁₀alkoxy, C₂₋₁₀alkenyloxy, C₂₋₁₀alkynyloxy, C₃₋₁₀cycloalkyl, heterocycloalkyl, aryl, heteroaryl, C₁₋₆alkylene-O—C₁₋₆alkyl, C₁₋₆alkylene-O—C₁₋₆haloalkyl, C₂₋₆alkenylene-O—C₁₋₆haloalkyl, C₂₋₆alkynylene-O—C₁₋₆haloalkyl, C₁₋₆alkylene-C₃₋₈cycloalkyl, C₁₋₆alkylene-heterocycloalkyl, C₁₋₆alkylene-aryl, C₁₋₆alkylene-heteroaryl, C₁₋₁₀alkyl-C(O)R¹, C₂₋₁₀alkenyl-C(O)R¹, C₂₋₁₀alkynyl-C(O)R¹, C₁₋₁₀haloalkyl-C(O)R¹, C₁₋₁₀cyanoalkyl-C(O)R¹, C₁₋₁₀alkoxy-C(O)R¹, C₂₋₁₀alkenyloxy-C(O)R¹, C₃₋₁₀cycloalkyl-C(O)R¹, heterocycloalkyl-C(O)R¹, aryl-C(O)R¹, heteroaryl-C(O)R¹, C₁₋₆alkylene-O—C₁₋₆alkyl-C(O)R¹, C₁₋₆alkylene-O—C₁₋₆haloalkyl-C(O)R¹, C₂₋₆alkenylene-O—C₁₋₆haloalkyl-C(O)R¹, C₂₋₆alkenylene-O—C₁₋₆haloalkyl-C(O)R¹, C₁₋₆alkylene-C₃₋₈cycloakyl-C(O)R¹, C₁₋₆alkylene-heterocycloalkyl-C(O)R¹, C₁₋₆alkylene-aryl-C(O)R¹, C₁₋₆alkylene-heteroaryl-C(O)R¹, C₁₋₁₀alkyl-OC(O)R¹, C₂₋₁₀alkenyl-OC(O)R¹, C₂₋₁₀alkynyl-OC(O)R¹, C₁₋₁₀haloalkyl-OC(O)R¹, C₁₋₁₀cyanoalkyl-OC(O)R¹, C₁₋₁₀alkoxy-OC(O)R¹, C₂₋₁₀alkenyloxy-OC(O)R¹, C₃₋₁₀cycloalkyl-OC(O)R¹, heterocycloalkyl-OC(O)R¹, aryl-OC(O)R¹, heteroaryl-OC(O)R¹, C₁₋₆alkylene-O—C₁₋₆alkyl-OC(O)R¹, C₁₋₆alkylene-O—C₁₋₆haloalkyl-OC(O)R¹, C₂₋₆alkenylene-O—C₁₋₆haloalkyl-O—C(O)R¹, C₂₋₆alkenylene-O—C₁₋₆haloalkyl-O—C(O)R¹, C₁₋₆alkylene-C₃₋₁₀cycloalkyl-O—C(O)R¹, C₁₋₆alkylene-heterocycloalkyl-O—C(O)R¹, C₁₋₆alkylene-aryl-O—C(O)R¹, C₁₋₆alkylene-heteroaryl-O—C(O)R¹, C₁₋₁₀alkyl-C(O)OR¹, C₂₋₁₀alkenyl-C(O)OR¹, C₂₋₁₀alkynyl-C(O)OR¹, C₁₋₁₀haloalkyl-C(O)OR¹, C₁₋₁₀cyanoalkyl-C(O)OR¹, C₁₋₁₀alkoxy-C(O)OR¹, C₂₋₁₀alkenyloxy-C(O)OR¹, C₃₋₁₀cycloalkyl-C(O)OR¹, heterocycloalkyl-C(O)OR¹, aryl-C(O)OR¹, heteroaryl-C(O)OR¹, C₁₋₆alkylene-O—C₁₋₆alkyl-C(O)OR¹, C₁₋₆alkylene-O—C₁₋₆haloalkyl-C(O)OR¹, C₂₋₆alkenylene-O—C₁₋₆haloalkyl-C(O)OR¹, C₂₋₆alkenylene-O—C₁₋₆haloalkyl-C(O)OR¹, C₁₋₆alkylene-C₃₋₈cycloakyl-C(O)OR¹, C₁₋₆alkylene-heterocycloalkyl-C(O)OR¹, C₁₋₆alkylene-aryl-C(O)OR¹, C₁₋₆alkylene-heteroaryl-C(O)OR¹, C₁₋₆alkylene-O—R¹, C₁₋₆alkylene-C(O)R¹, C₁₋₆alkylene-O—C(O)R¹, C₁₋₆alkylene-C(O)OR¹, C₁₋₆alkylene-O—C(O)OR¹, C₁₋₆alkyleneNR¹R², C₁₋₆alkylene-NR²R¹, C₁₋₆alkylene-C(O)NR¹R², C₁₋₆alkylene-NR¹C(O)R², C₁₋₆alkylene-NR¹C(O)NR³R², C₁₋₆alkylene-S—R¹, C₁₋₆alkylene-S(O)R¹, C₁₋₆alkylene-SO₂R¹, C₁₋₆alkylene-SO₂NR¹R², C₁₋₆alkylene-NR¹SO₂R², C₁₋₆alkylene-NR³SO₂NR¹R², C(O)NR¹R² and C₁₋₆alkylene-NR¹C(O)OR², wherein R is optionally substituted with C₁₋₄alkyl and any cyclic or heterocyclic moiety is optionally fused to a further cyclic or heterocyclic moiety; and R¹ and R² are each independently selected from the group consisting of H, C₁₋₆alkyl, C₁₋₆haloalkyl, C₂₋₆alkenyl, C₂₋₆alkynyl, C₃₋₁₀cycloalkyl, C₁₋₆alkylene-C₃₋₁₀cycloalkyl, heterocycloalkyl, aryl, C₁₋₆alkylene-aryl, C₁₋₆alkylene-heterocycloalkyl, heteroaryl, and C₁₋₆alkylene-heteroaryl, wherein any cyclic or heterocyclic moiety is optionally fused to a further cyclic and heterocyclic moieties.

In an embodiment, R is selected from D/L-amino acids, C₁₋₆alkyl, C₂₋₆alkenyl, C₂₋₆alkynyl, C₁₋₆haloalkyl, C₁₋₆cyanoalkyl, C₁₋₆alkoxy, C₂₋₆alkenyloxy, C₂₋₆alkynyloxy, C₃₋₆cycloalkyl, heterocycloalkyl, aryl, heteroaryl, C₁₋₄alkylene-O—C₁₋₄alkyl, C₁₋₄alkylene-O—C₁₋₄haloalkyl, C₂₋₄alkenylene-O—C₁₋₄haloalkyl, C₂₋₄alkynylene-O—C₁₋₄haloalkyl, C₁₋₄alkylene-C₃₋₆cycloalkyl, C₁₋₄alkylene-heterocycloalkyl, C₁₋₄alkylene-aryl, C₁₋₄alkylene-heteroaryl, C₁₋₆alkyl-C(O)R¹, C₂₋₆alkenyl-C(O)R¹, C₂₋₆alkynyl-C(O)R¹, C₁₋₆haloalkyl-C(O)R¹, C₁₋₆cyanoalkyl-C(O)R¹, C₁₋₆alkoxy-C(O)R¹, C₂₋₆alkenyloxy-C(O)R¹, C₃₋₆cycloalkyl-C(O)R¹, heterocycloalkyl-C(O)R¹, aryl-C(O)R¹, heteroaryl-C(O)R¹, C₁₋₄alkylene-O—C₁₋₄alkyl-C(O)R¹, C₁₋₄alkylene-O—C₁₋₄haloalkyl-C(O)R¹, C₂₋₄alkenylene-O—C₁₋₄haloalkyl-C(O)R¹, C₂₋₄alkenylene-O—C₁₋₄haloalkyl-C(O)R¹, C₁₋₄alkylene-C₃₋₆cycloalkyl-C(O)R¹, C₁₋₄alkylene-heterocycloalkyl-C(O)R¹, C₁₋₄alkylene-aryl-C(O)R¹, C₁₋₄alkylene-heteroaryl-C(O)R¹, C₁₋₆alkyl-OC(O)R¹, C₂₋₆alkenyl-OC(O)R¹, C₂₋₆alkynyl-OC(O)R¹, C₁₋₆haloalkyl-OC(O)R¹, C₁₋₆cyanoalkyl-OC(O)R¹, C₁₋₆alkoxy-OC(O)R¹, C₂₋₆alkenyloxy-OC(O)R¹, C₃₋₆cycloalkyl-OC(O)R, heterocycloalkyl-OC(O)R, aryl-OC(O)R¹, heteroaryl-OC(O)R¹, C₁₋₄alkylene-O—C₁₋₄alkyl-OC(O)R¹, C₁₋₄alkylene-O—C₁₋₄haloalkyl-OC(O)R¹, C₂₋₄alkenylene-O—C₁₋₄haloalkyl-O—C(O)R¹, C₂₋₄alkenylene-O—C₁₋₄haloalkyl-O—C(O)R¹, C₁₋₄alkylene-C₃₋₆cycloalkyl-O—C(O)R¹, C₁₋₄alkylene-heterocycloalkyl-O—C(O)R, C₁₋₄alkylene-aryl-O—C(O)R¹, C₁₋₄alkylene-heteroaryl-O—C(O)R¹, C₁₋₆alkyl-C(O)OR¹, C₂₋₆alkenyl-C(O)OR¹, C₂₋₆alkynyl-C(O)OR¹, C₁₋₆haloalkyl-C(O)OR¹, C₁₋₆cyanoalkyl-C(O)OR¹, C₁₋₆alkoxy-C(O)OR¹, C₂₋₆alkenyloxy-C(O)OR¹, C₃₋₆cycloalkyl-C(O)OR¹, heterocycloalkyl-C(O)OR¹, aryl-C(O)OR¹, heteroaryl-C(O)OR¹, C₁₋₄alkylene-O—C₁₋₄alkyl-C(O)OR¹, C₁₋₄alkylene-O—C₁₋₄haloalkyl-C(O)OR¹, C₂₋₄alkenylene-O—C₁₋₄haloalkyl-C(O)OR¹, C₂₋₄alkenylene-O—C₁₋₄haloalkyl-C(O)OR¹, C₁₋₄alkylene-C₃₋₆cycloalkyl-C(O)OR¹, C₁₋₄alkylene-heterocycloalkyl-C(O)OR¹, C₁₋₄alkylene-aryl-C(O)OR¹, C₁₋₄alkylene-heteroaryl-C(O)OR¹, C₁₋₄alkylene-O—R¹, C₁₋₄alkylene-C(O)R¹, C₁₋₄alkylene-O—C(O)R¹, C₁₋₄alkylene-C(O)OR¹, C₁₋₄alkylene-O—C(O)OR¹, C₁₋₄alkyleneNR¹R², C₁₋₄alkylene-NR²R¹, C₁₋₄alkylene-C(O)NR¹R², C₁₋₄alkylene-NR¹C(O)R², C₁₋₄alkylene-NR¹C(O)NR³R², C₁₋₄alkylene-S—R¹, C₁₋₄alkylene-S(O)R¹, C₁₋₄alkylene-SO₂R¹, C₁₋₄alkylene-SO₂NR¹R², C₁₋₄alkylene-NR¹SO₂R², C₁₋₄alkylene-NR³SO₂NR¹R², C(O)NR¹R² and C₁₋₄alkylene-NR¹C(O)OR², wherein R is optionally substituted with C₁₋₄alkyl and any cyclic or heterocyclic moiety is optionally fused to a further cyclic or heterocyclic moiety.

In an embodiment, R is selected from D/L-amino acids and C₁₋₆alkylene-NR¹R². In another embodiment, the D/L amino acids is selected from serine and threonine. In a further embodiment, R is C₁₋₄alkylene-NR¹R².

In an embodiment, R¹ and R² are each independently selected from the group consisting of H, C₁₋₄alkyl, C₁₋₄haloalkyl, C₂₋₄alkenyl, C₂₋₄alkynyl, C₃₋₆cycloalkyl, C₁₋₄alkylene-C₃₋₆cycloalkyl, heterocycloalkyl, aryl, C₁₋₄alkylene-aryl, C₁₋₄alkylene-heterocycloalkyl, heteroaryl, and C₁₋₄alkylene-heteroaryl, wherein any cyclic or heterocyclic moiety is optionally fused to a further cyclic or heterocyclic moiety. In another embodiment, R¹ and R² are selected from H and C₁₋₄alkyl.

In an embodiment of the application, the conditions to provide a compound of Formula (III) comprise reacting a compound of Formula (II) with Vilsmeier reagent. In an embodiment, the Vilsmeier reagent is generated in situ in inert solvents at temperature and time sufficient for the conversion to proceed to a sufficient extent. The Vilsmeier reagent is the reaction product of a substituted amide with an oxychloride to provide a substituted chloroimminium ion. In an embodiment of the present application, the Vilsmeier reagent is generated in situ from DMF and oxalyl chloride. Examples of non limiting reaction temperatures include, but are not limited to, −20° C. to about 10° C. or −5° C. to about 5° C. Examples of non-limiting reaction times are about 5 minutes to about 1 hour or about 15 minutes to about 30 minutes. Examples of non limiting inert solvents include, but are not limited to halogenated solvents. In an embodiment, the halogenated solvent is dichloromethane.

In an embodiment, the conditions to provide a compound of Formula (III) further comprises the addition of a sulfurating reagent such as hydrogen sulfide (H₂S) in the presence of pyridine or the equivalent suitable salt of hydrogen sulfide (such as NaSH) is added to the reaction mixture in an inert solvent at temperatures and times sufficient to proceed to a sufficient extent. In an embodiment, the solution comprising the suitable salt of hydrogen sulfide must be added to the reaction mixture quickly, with vigorous stirring to allow the conversion to proceed to a sufficient extent. In another embodiment, the solution comprising the suitable salt of hydrogen sulfide must be as concentrated as possible. Examples of non-limiting reaction temperatures include, but are not limited to, −40° C. to about 10° C., −30° C. to about 5° C. or −20° C. to about −10° C. Examples of non-limiting reaction times include, but are not limited to 5 minutes to about 1 hour or about 15 minutes to about 30 minutes. In an embodiment, the inert solvent is an aqueous solvent. In a further embodiment, the aqueous solvent is water.

In an embodiment, the conditions to provide the compound of Formula (I) comprises reacting the compound of Formula (III) with 2,2-difluoro-1,3-dimethylimidazoline in inert solvents at temperature and time sufficient for the conversion to proceed to a sufficient extent. Examples of non-limiting temperatures include, but are not limited to, −10° C. to about 100° C., −5° C. to about 50° C. or 0° C. to about 30° C. Examples of non-limiting reaction times include, but are not limited to 5 minutes to about 10 hours, 15 minutes to about 5 hours or about 30 minutes to about 3 hours. Examples of non-limiting inert solvents, include but are not limited to organic solvents. In an embodiment, the inert solvent is acetonitrile.

In an embodiment, 2,2-difluoro-1,3-dimethylimidazoline is generated in situ from 2-chloro-1,3-dimethyl-4,5-dihydroimidazol-1-ium chloride and potassium fluoride in an inert solvent at temperature and time sufficient for the conversion to proceed to a sufficient extent. Examples of non-limiting temperatures include, but are not limited to, 10° C. to about 120° C., 50° C. to about 100° C. or 70° C. to about 90° C. Examples of non-limiting reaction times include, but are not limited to 5 hours to about 30 hours or about 10 hours to about 20 hours. Examples of non-limiting inert solvents, include but are not limited to organic solvents. In an embodiment, the inert solvent is acetonitrile.

In an embodiment, the compound of Formula (I) is selected from:

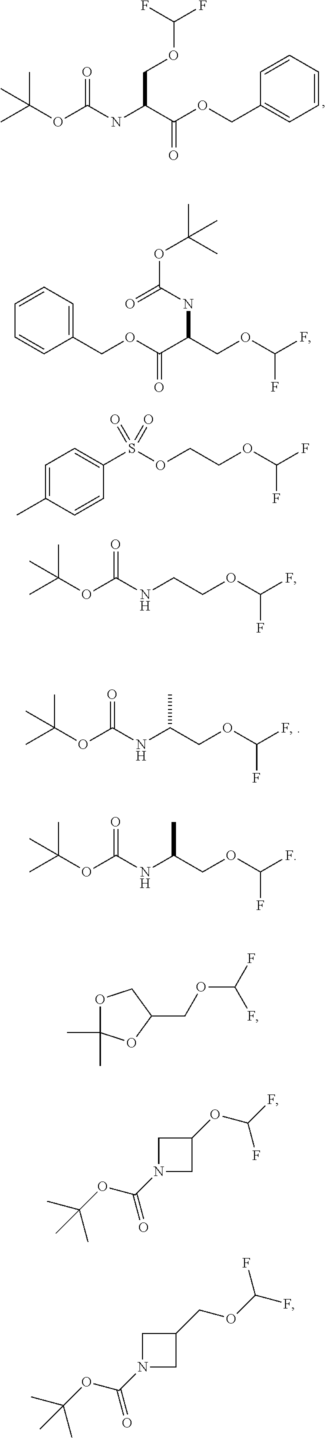

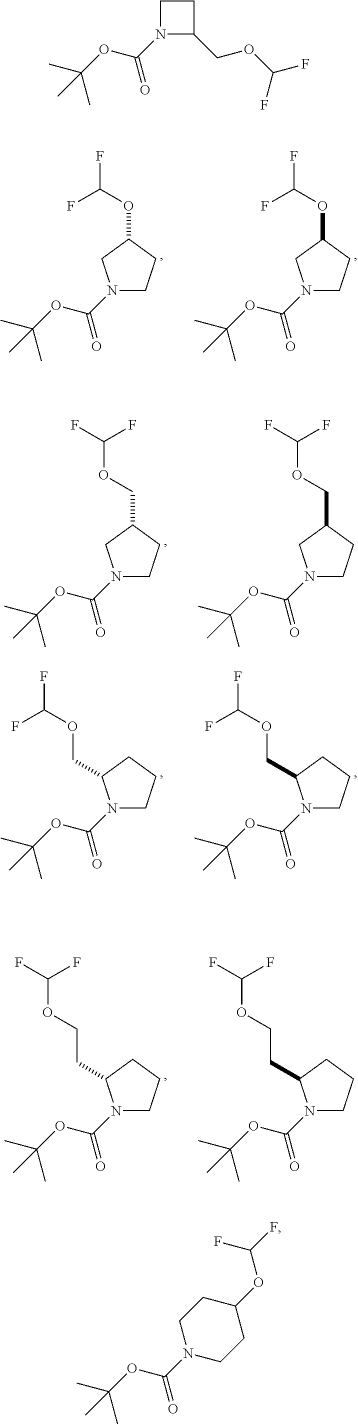

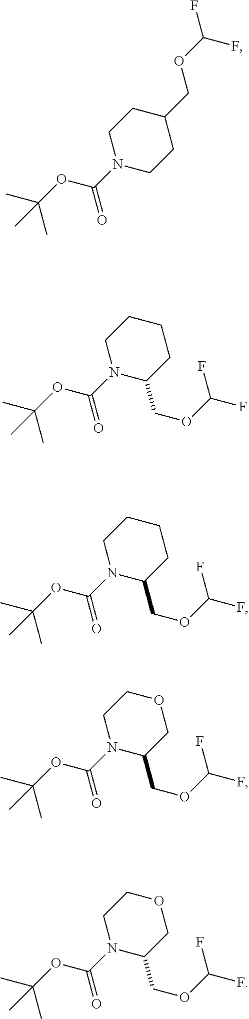 [SMILES: CC(C)(C)OC(=O)N1CCC(COC(F)F)CC1 ; CC(C)(C)OC(=O)N1CCCC[C@@H]1COC(F)F ; CC(C)(C)OC(=O)N1CCCC[C@H]1COC(F)F ; CC(C)(C)OC(=O)N1CCOC[C@H]1COC(F)F ; CC(C)(C)OC(=O)N1CCOC[C@@H]1COC(F)F]

### III. Compounds and Compositions of the Application

Difluoromethyl ether compounds of the present application of the Formula (I) were prepared.

In one aspect, the present application includes a compound of Formula (I) or a pharmaceutically acceptable salt, solvate and/or prodrug thereof.

Accordingly, the present application includes a compound of Formula (I) or a pharmaceutically acceptable salt, solvate and/or prodrug thereof:

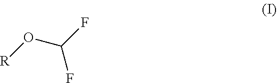

wherein:
R is selected from the group consisting of C₁₋₁₀alkyl, C₂₋₁₀alkenyl, C₂₋₁₀alkynyl, C₁₋₁₀haloalkyl, C₁₋₁₀cyanoalkyl, C₁₋₁₀alkoxy, C₂₋₁₀alkenyloxy, C₂₋₁₀alkynyloxy, C₃₋₁₀cycloalkyl, heterocycloalkyl, aryl, heteroaryl, C₁₋₆alkylene-O—C₁₋₆alkyl, C₁₋₆alkylene-O—C₁₋₆haloalkyl, C₂₋₆alkenylene-O—C₁₋₆haloalkyl, C₂₋₆alkynylene-O—C₁₋₆haloalkyl, C₁₋₆alkylene-C₃₋₈cycloalkyl, C₁₋₆alkylene-heterocycloalkyl, C₁₋₆alkylene-aryl, C₁₋₆alkylene-heteroaryl, C₁₋₁₀alkyl-C(O)R¹, C₂₋₁₀alkenyl-C(O)R¹, C₂₋₁₀alkynyl-C(O)R¹, C₁₋₁₀haloalkyl-C(O)R¹, C₁₋₁₀cyanoalkyl-C(O)R¹, C₁₋₁₀alkoxy-C(O)R¹, C₂₋₁₀alkenyloxy-C(O)R¹, C₃₋₁₀cycloalkyl-C(O)R¹, heterocycloalkyl-C(O)R¹, aryl-C(O)R¹, heteroaryl-C(O)R¹, C₁₋₆alkylene-O—C₁₋₆alkyl-C(O)R¹, C₁₋₆alkylene-O—C₁₋₆haloalkyl-C(O)R¹, C₂₋₆alkenylene-O—C₁₋₆haloalkyl-C(O)R¹, C₂₋₆alkenylene-O—C₁₋₆haloalkyl-C(O)R¹, C₁₋₆alkylene-C₃₋₈cycloalkyl-C(O)R¹, C₁₋₆alkylene-heterocycloalkyl-C(O)R¹, C₁₋₆alkylene-aryl-C(O)R¹, C₁₋₆alkylene-heteroaryl-C(O)R¹, C₁₋₁₀alkyl-OC(O)R¹, C₂₋₁₀alkenyl-OC(O)R¹, C₂₋₁₀alkynyl-OC(O)R¹, C₁₋₁₀haloalkyl-OC(O)R¹, C₁₋₁₀cyanoalkyl-OC(O)R¹, C₁₋₁₀alkoxy-OC(O)R¹, C₂₋₁₀alkenyloxy-OC(O)R¹, C₃₋₁₀cycloalkyl-OC(O)R¹, heterocycloalkyl-OC(O)R¹, aryl-OC(O)R¹, heteroaryl-OC(O)R¹, C₁₋₆alkylene-O—C₁₋₆alkyl-OC(O)R¹, C₁₋₆alkylene-O—C₁₋₆haloalkyl-OC(O)R¹, C₂₋₆alkenylene-O—C₁₋₆haloalkyl-OC(O)R¹, C₂₋₆alkenylene-O—C₁₋₆haloalkyl-OC(O)R¹, C₁₋₆alkylene-C₃₋₈cycloalkyl-OC(O)R¹, C₁₋₆alkylene-heterocycloalkyl-OC(O)R¹, C₁₋₆alkylene-aryl-OC(O)R¹, C₁₋₆alkylene-heteroaryl-OC(O)R¹, C₁₋₁₀alkyl-C(O)OR¹, C₂₋₁₀alkenyl-C(O)OR¹, C₂₋₁₀alkynyl-C(O)OR¹, C₁₋₁₀haloalkyl-C(O)OR¹, C₁₋₁₀cyanoalkyl-C(O)OR¹, C₁₋₁₀alkoxy-C(O)OR¹, C₂₋₁₀alkenyloxy-C(O)OR¹, C₃₋₁₀cycloalkyl-C(O)OR¹, heterocycloalkyl-C(O)OR¹, aryl-C(O)OR¹, heteroaryl-C(O)OR¹, C₁₋₆alkylene-O—C₁₋₆alkyl-C(O)OR¹, C₁₋₆alkylene-O—C₁₋₆haloalkyl-C(O)OR¹, C₂₋₆alkenylene-O—C₁₋₆haloalkyl-C(O)OR¹, C₂₋₆alkenylene-O—C₁₋₆haloalkyl-C(O)OR¹, C₁₋₆alkylene-C₃₋₈cycloalkyl-C(O)OR¹, C₁₋₆alkylene-heterocycloalkyl-C(O)OR¹, C₁₋₆alkylene-aryl-C(O)OR¹, C₁₋₆alkylene-heteroaryl-C(O)OR¹, C₁₋₆alkylene-O—R¹, C₁₋₆alkylene-C(O)R¹, C₁₋₆alkylene-O—C(O)R¹, C₁₋₆alkylene-C(O)OR¹, C₁₋₆alkylene-O—C(O)OR¹, C₁₋₆alkylene-NR²R¹, C₁₋₆alkylene-C(O)NR¹R², C₁₋₆alkylene-NR¹C(O)R², C₁₋₆alkylene-NR¹C(O)NR³R², C₁₋₆alkylene-S—R¹, C₁₋₆alkylene-S(O)R¹, C₁₋₆alkylene-SO₂R¹, C₁₋₆alkylene-SO₂NR¹R², C₁₋₆alkylene-NR¹SO₂R², C₁₋₆alkylene-NR³SO₂NR¹R², C(O)NR¹R² and C₁₋₆alkylene-NR¹C(O)OR², wherein R is optionally substituted with C₁₋₄alkyl and any cyclic or heterocyclic moiety is optionally fused to a further cyclic or heterocyclic moiety;

In an embodiment, R¹ and R² are each independently selected from the group consisting of H, C₁₋₆alkyl, C₁₋₆haloalkyl, C₂₋₆alkenyl, C₂₋₆alkynyl, C₃₋₁₀cycloalkyl, C₁₋₆alkylene-C₃₋₁₀cycloalkyl, heterocycloalkyl, aryl, C₁₋₆alkylene-aryl, C₁₋₆alkylene-heterocycloalkyl, heteroaryl, and C₁₋₆alkylene-heteroaryl, wherein any cyclic or heterocyclic moiety is optionally fused to a further cyclic or heterocyclic moiety.

In an embodiment, the compound of Formula (I) is in the form of a pharmaceutically acceptable salt, solvate and/or prodrug thereof.

In an embodiment, a pharmaceutical composition comprising one or more compounds of Formula (I) as defined in the application or a pharmaceutically acceptable salt, and/or solvate thereof, and a pharmaceutically acceptable carrier and/or diluent.

Preparation of Compounds of the Application

Compounds of the present application are prepared by a controlled and scalable synthetic process. Some starting materials for preparing of given compound of Formula (I) are available from commercial chemical sources. Other starting materials are readily prepared from available precursors using straightforward transformations that are well known in the art.

In an embodiment, the compounds of Formula (I) represented for example, by compound (3) are generally prepared according to the process illustrated in Scheme 5. Variables in the following schemes are as defined above for the compounds of Formula (I) unless otherwise specified.

### A. General Methods

All starting materials used herein were commercially available or earlier described in the literature. The ¹H and ¹³C NMR spectra were recorded either on Bruker 300, Bruker DPX400 or Varian +400 spectrometers operating at 300, 400 and 400 MHz for ¹H NMR respectively, using TMS or the residual solvent signal as an internal reference, in deuterated chloroform as solvent unless otherwise indicated. All reported chemical shifts are in ppm on the delta-scale, and the fine splitting of the signals appearing in the recordings is generally indicated, for example as s: singlet, br s: broad singlet, d: doublet, t: triplet, q: quartet, m: multiplet. Unless otherwise indicated in the tables below ¹H NMR data was obtained at 300 MHz, using CDCl₃ as the solvent.

Purification of products was carried out using Chem Elut Extraction Columns (Varian, cat #1219-8002), Mega BE-SI (Bond Elut Silica) SPE Columns (Varian, cat #12256018; 12256026; 12256034) or by flash chromatography in silica-filled glass columns.

### B. Synthesis and Characterization of Compounds

Scheme 5 outlines the synthesis of an examplary compound of Formula (I), wherein R—OH is benzyl (2S)-2-(tert-butoxycarbonylamino)-3-hydroxy-propanoate.

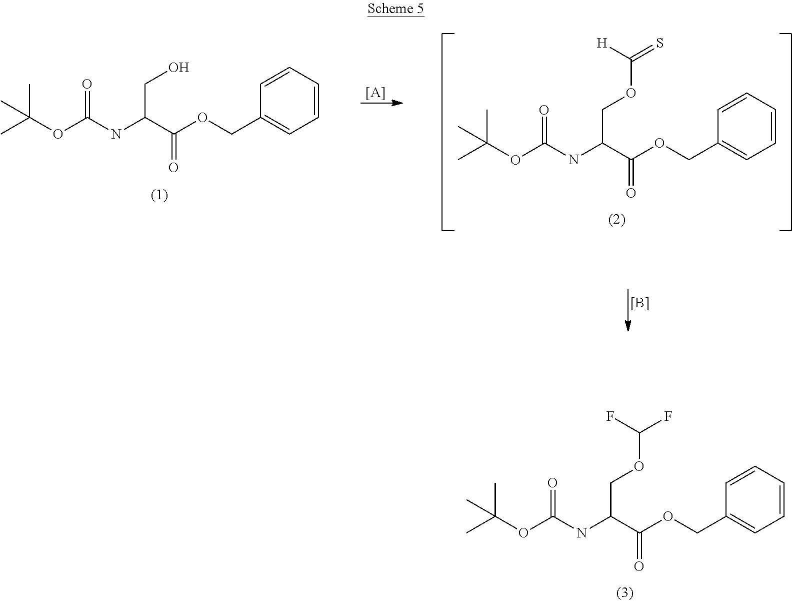

Reagents and conditions used in Scheme 5: [A] (i) (COCl)₂, DMF in CH₂Cl₂, 0° C. (ii) NaSH, THF, 0° C./30 min, [B] 2,2-difluoro-1,3-dimethyl-imidazolidine, CH₂Cl₂ 0° C./1 hr.

### Preparation of (S)-2-tert-Butoxycarbonylamino-3-thioformyloxy-propionic acid benzyl ester (2)

In a 3 L round bottom flask equipped with a stir bar was added DMF (67 mL, 0.868 mol), and dichloromethane (1.2 L). Then oxalyl chloride (78 mL, 0.868 mol) slowly at 0° C. The reaction mixture was stirred for 30 min after the addition of oxalyl chloride was over. Then (S)-2-tert-Butoxycarbonylamino-3-hydroxy-propionic acid benzyl ester (1) (171 g, 0.579 mol) was added portionwise as solid (or with THF) to the reaction mixture, and then stirred for an additional 30 min. The mixture was then cooled to −15° C. and treated with NaSH (4 eq) in ice water (˜40 mL). The organic layer separated and dried over MgSO₄ and concentrated in vacuo. The isolated crude residue was filtered on silica-gel with and washed with 4% to 5% ethyl acetate and 5% DCM in hexanes, to give the desired product (2) as off-white yellowish powder (176, 90%). ¹H NMR (300 MHz, CDCl₃): δ (ppm) 9.58 (s, 1H), 7.25 (m, 5H), 5.35 (broad s, 1H), 5.15 (m, 3H), 4.80 (m, 2H), 1.35 (s, 9H).

The solution of NaSH must be added to the reaction mixture quickly, with vigorous stirring in order to obtain good yields, and to minimize side products.

The solution of NaSH should be as concentrated as possible.

Hydrogen sulfide gas can be used as sulfide source instead of an aqueous solution of NaSH.

The crude product can be used without any extraction or purification, and the yield is quantitative as determined the next step.

### Preparation of (S)-2-tert-Butoxycarbonylamino-3-difluoromethoxy-propionic acid benzyl ester (3)

To a solution of (S)-2-tert-Butoxycarbonylamino-3-thioformyloxy-propionic acid benzyl ester (2) (100 g, 294.5 mmol) in dichloromethane (750 mL) at 0° C., was added 2,2-Difluoro-1,3-dimethyl-imidazolidine (50.0 g, 353.5 mmol) with stirring. After 30 min., the reaction mixture was concentrated onto silica gel and purified by silica-gel column chromatography, eluting with 7.5% to 10% ethyl acetate in hexanes, to provide (S)-2-tert-Butoxycarbonylamino-3-difluoromethoxy-propionic acid benzyl ester (3) (102.05 g, 100%) as a colorless sticky oil. ¹H NMR (300 MHz, CDCl₃): δ (ppm) 7.32-7.38 (m, 5H), 6.18 (wt, 1H), 5.28 (dd, 1H), 5.19 (dd, 2H), 4.55 (dt, 1H) 4.22 (td, 1H), 4.15 (m, 2H), 1.38 (s, 9H).

### C. One Pot Difluoromethylation Process

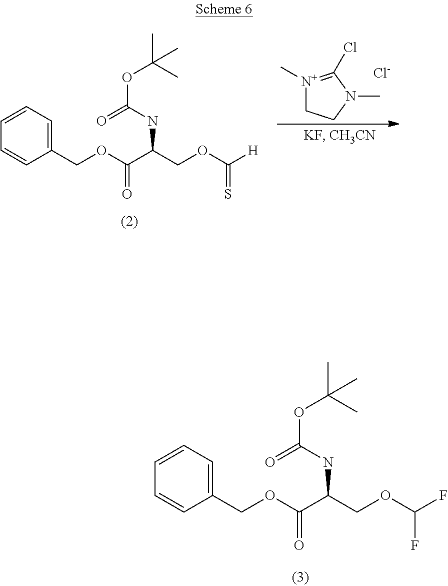

### Preparation of (S)-2-tert-Butoxycarbonylamino-3-difluoromethoxy-propionic acid benzyl ester (3)

2-chloro-1,3-dimethyl-4,5-dihydroimidazol-1-ium chloride (40 g, 236 mmol) and KF (spray dried, 54.4 g, 937 mmol) were stirred in acetonitrile (200 mL) under nitrogen at gentle reflux (80-90° C. oil bath) overnight (16-20 hr). The mixture was then cooled to 0° C. and treated with (S)-benzyl 2-((tert-butoxycarbonyl)amino)-3-(thioformyloxy)propanoate (36.8 g, 108.5 mmol) as a solution in DCM (50 mL) dropwise over a period of 0.5 h. Upon completion of the addition, the mixture was warmed to room temperature and stirred for an additional 2 h. The mixture was then filtered to remove the inorganic salts. The filtrate (Note 1) was diluted with diethyl ether and washed with brine (1×), water (4×) and brine (1×). (Note 2) The organic phase was dried, filtered and concentrated then chromatographed in 0-30% ethyl acetate in hexanes. The product containing fractions were concentrated in vacuo to give the desired product as a pale yellow oil (35.8 g, 95%).

Note 1:

The filtrate was concentrated. (in plant setting, the easy removal of acetonitrile allows for the recycling of the solvent resulting in effective cost savings).

Note 2:

In the plant setting, multiple washings are typically avoided unless absolutely necessary. Hence, once the acetonitrile is removed only one wash with brine is required.

### Preparation of tert-butyl N-(2-hydroxyethyl)carbamate

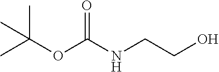

To a stirred solution of ethanolamine (5 g, 4.94 mL, 81.8 mmol) and sodium hydroxide (327 mg, 8.18 mmol) in water (30 mL) was added di-tert-butyl dicarbonate (19.65 g, 90.0 mmol) as a solution in tetrahydrofuran (30 mL). The mixture was stirred overnight at room temperature (mild exotherm, steady bubbling observed). The mixture was diluted with diethyl ether and washed with brine (2×), water (2×) and brine (1×). The organic phase was dried, filtered and concentrated in vacuo then chromatographed in 25-75% ethyl acetate in hexanes. The product containing fractions were concentrated in vacuo giving a thick colourless syrup (11.06 g, 83%).

### Preparation of O-[2-(tert-butoxycarbonylamino)ethyl] methanethioate

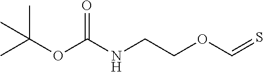

To a stirred solution of DMF (4.80 mL, 62.0 mmol) in DCM (75 mL) cooled to −20° C. under nitrogen was added oxalyl chloride (5.32 mL, 62.0 mmol) slowly over a period of 30 min (bubbling observed). The mixture was stirred for a further 15 min then treated with tert-butyl N-(2-hydroxyethyl)carbamate (5 g, 31.0 mmol) as a solution in DCM (10 mL). The mixture was stirred for a further 10 min (at −20° C.) then treated with NaHS (7.4 g) as a solution in water (10 mL, quickly, with vigorous stirring) then warmed to room temperature. The mixture was diluted with water and the organic phase was washed with water (1×) and brine (1×). The organic phase was dried, filtered and concentrated in vacuo then chromatographed in 0-15% ethyl acetate in hexanes. The product containing fractions were concentrated in vacuo giving a yellow oil (5.36 g, 84%). ¹H NMR (CDCl3, 300 MHz) δ 9.72 (s, 1H), 4.80 (brs, 1H), 4.59-4.55 (m, 2H), 3.60-3.51 (m, 2H), 1.45 (s, 9H).

### Preparation of tert-butyl N-[2-(difluoromethoxy)ethyl]carbamate

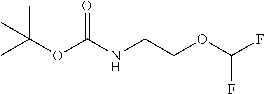

2-chloro-1,3-dimethyl-4,5-dihydroimidazol-1-ium chloride (8.5 g, 50 mmol) and KF (12.8 g, 220 mmol) were combined with ACN (100 mL) and stirred at reflux temperature overnight. The mixture was then cooled to room temperature and treated with O-[2-(tert-butoxycarbonylamino)ethyl]methanethioate (5.3 g, 25.8 mmol) as a solution in DCM (10 mL). The resulting mixture was stirred for 2 h. The mixture was diluted with diethyl ether and washed with brine (2×) water (2×) and brine (1×). The organic phase was dried, filtered and concentrated in vacuo then chromatographed in 0-30% ethyl acetate in hexanes. The product containing fractions were concentrated in vacuo giving the desired product as a clear oil (5.1 g, 93%). ¹H NMR (d₆-DMSO, 300 MHz) δ 6.81, (brs, 1H), 6.62 (t, J=75 Hz, 1H), 3.75-3.54 (m, 4H), 1.37 (s, 9H).

### Preparation of tert-butyl N-[(1R)-2-hydroxy-1-methyl-ethyl]carbamate

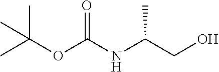

To a stirred solution of D-alaninol (9.5 g, 126 mmol) and sodium hydroxide (506 mg, 12.6 mmol) in water (100 mL) was added di-tert-butyl dicarbonate (30.3 g, 139 mmol) as a solution in THF (100 mL). The resulting mixture was stirred at room temperature overnight (steady bubbling observed). The mixture was diluted with diethyl ether and washed with brine (2×), water (2×) and brine (1×). The organic phase was dried, filtered and concentrated in vacuo. The residue was stirred in hexanes. The resulting suspension was filtered to collect the desired product as a white solid (18.56 g, 84%)

### Preparation of tert-butyl N-[(1R)-2-hydroxy-1-methyl-ethyl]carbamate

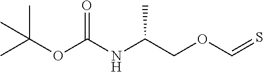

To a stirred solution of DMF (4.42 mL, 57.1 mmol) in DCM (50 mL) cooled to −20° C. under nitrogen was added oxalyl chloride (4.89 mL, 57.1 mmol) slowly over a period of 30 min (bubbling observed). The mixture was stirred for a further 15 min then treated with tert-butyl N-[(1R)-2-hydroxy-1-methyl-ethyl]carbamate (5 g, 28.5 mmol) as a solution in DCM (10 mL). The mixture was stirred for a further 10 min (at −20° C.) then treated with NaHS (6 g) as a solution in water (10 mL, quickly, with vigorous stirring) then warmed to room temperature. The mixture was diluted with water and the organic phase was washed with water (1×) and brine (1×). The organic phase was dried, filtered and concentrated in vacuo then chromatographed in 0-15% ethyl acetate in hexanes. The product containing fractions were concentrated in vacuo giving a yellow oil which slowly solidified (5.34 g, 85%). ¹H NMR (CDCl₃, 300 MHz) δ 9.74 (s, 1H), 4.56 (brs, 1H), 4.49-4.42 (m, 2H), 4.20-4.10 (m, 1H), 1.45 (s, 9H), 1.24 (d, J=3 Hz, 3H).

### Preparation of tert-butylN-[(1R)-2-(difluoromethoxy)-1-methyl-ethyl]carbamate

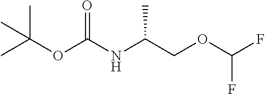

2-chloro-1,3-dimethyl-4,5-dihydroimidazol-1-ium chloride (8.5 g, 50 mmol) and KF (12.8 g, 220 mmol) were combined with ACN (100 mL) and stirred at reflux temperature overnight. The mixture was then cooled to room temperature and treated with O-[(2R)-2-(tert-butoxycarbonylamino)propyl]methanethioate (5.3 g, 24.1 mmol) as a solution in DCM (10 mL). The resulting mixture was stirred for 2 h. The mixture was diluted with diethyl ether and washed with brine (2×) water (2×) and brine (1×). The organic phase was dried, filtered and concentrated in vacuo then chromatographed in 0-30% ethyl acetate in hexanes. The product containing fractions were concentrated in vacuo giving the desired product as a clear oil (5.36 g, 98%). ¹H NMR (d₆-DMSO, 300 MHz) δ 6.83, (brs, 1H), 6.63 (t, J=76 Hz, 1H), 3.70-3.55 (m, 3H), 1.36 (s, 9H), 1.01 (d, J=3 Hz, 3H).

### Preparation of tert-butyl N-[(1S)-2-(difluoromethoxy)-1-methyl-ethyl]carbamate

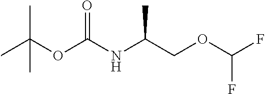

2-chloro-1,3-dimethyl-4,5-dihydroimidazol-1-ium chloride (8.5 g, 50 mmol) and KF (12.8 g, 220 mmol) were combined with ACN (100 mL) and stirred at reflux temperature overnight. The mixture was then cooled to room temperature and treated with O-[(2S)-2-(tert-butoxycarbonylamino)propyl]methanethioate (5 g, 22.7 mmol) as a solution in DCM (10 mL). The resulting mixture was stirred for 2 h. The mixture was diluted with diethyl ether and washed with brine (2×) water (2×) and brine (1×). The organic phase was dried, filtered and concentrated in vacuo then chromatographed in 0-30% ethyl acetate in hexanes. The product containing fractions were concentrated in vacuo giving the desired product as a clear oil (5.01 g, 97%). 1H NMR (d6-DMSO, 300 MHz) δ 6.83, (brs, 1H), 6.63 (t, J=76 Hz, 1H), 3.70-3.55 (m, 3H), 1.36 (s, 9H), 1.01 (d, J=3 Hz, 3H).

### Synthesis of 2-(difluoromethoxy)ethyl 4-methylbenzenesulfonate

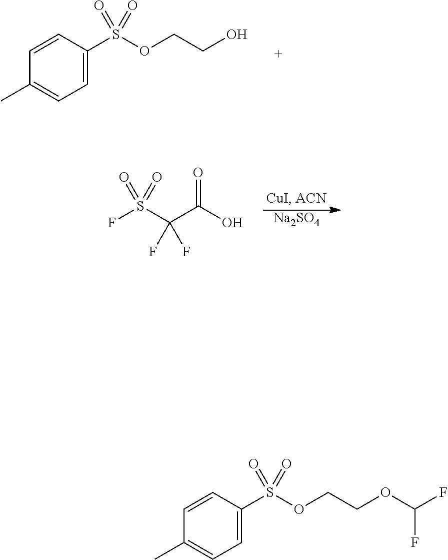

To a stirred solution of 2-hydroxyethyl 4-methylbenzenesulfonate (5.52 g, 25.5 mmol) in acetonitrile (40 mL) was added copper (I) iodide (972 mg, 5.1 mmol). The resulting mixture was stirred at 70° C. and treated with 2,2-difluoro-2-fluorosulfonyl-acetic acid as a solution in acetonitrile (5 mL) dropwise over a period of 30 min (mixture gradually turns dark red). The resulting mixture was treated with anhydrous sodium sulfate (5 mg) and stirring continued (steady evolution of gas observed, colour fades to yellow) for a further 30 min. The mixture was then cooled to room temperature, diluted with diethyl ether and washed with brine (1×), a 1:1 mixture of brine:water (2×) and brine (1×). The organic phase was dried over anhydrous sodium sulfate, filtered and concentrated in vacuo then chromatographed in 0-20% ethyl acetate in hexanes. The product containing fractions were concentrated in vacuo giving the desired product as a clear liquid (4.2 g, 62%).

Throughout the processes described herein, it is to be understood that, where appropriate, suitable protecting groups will be added to, and subsequently removed from, the various reactants and intermediates in a manner that will be readily understood by one skilled in the art. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to one skilled in the art. Examples of transformations are given herein, and it is to be understood that the described transformations are not limited only to the generic groups or substituents for which the transformations are exemplified. References and descriptions of other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or “Organic Synthesis”, Smith, McGraw Hill, (1994). Techniques for purification of intermediates and final products include for example, straight and reversed phase chromatography on column or rotating plate, recrystallisation, distillation and liquid-liquid or solid-liquid extraction, which will be readily understood by one skilled in the art.

While the present application has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A process for the preparation of difluoromethyl ethers comprising:
a) reacting a suitable alcohol with Vilsmeier reagent followed by a sulfurating reagent under conditions to provide a thioformyl ester; and
b) reacting the thioformyl ester of step (a) with 2,2-difluoro-1,3-dimethylimidazolidine under conditions to provide the difluoromethyl ether.

2. The process of claim 1, wherein the suitable alcohol is any suitable organic alcohol comprising carbon and hydrogen atoms, wherein 1 or more carbon atoms are optionally replaced with P, N, O and/or S.

3. The process of claim 2, wherein the suitable organic alcohol is any alcohol that is compatible reacting with the Vilsmeier reagent.

4. The process of claim 1, wherein the Vilsmeier reagent is generated in situ from DMF and oxalyl chloride.

5. The process of claim 1, wherein the sulfurating reagent comprises hydrogen sulfide (H₂S) in the presence of pyridine or is NaSH.

6. The process of claim 1, wherein the 2,2-difluoro-1,3-dimethylimidazoline is generated in situ from 2-chloro-1,3-dimethyl-4,5-dihydroimidazol-1-ium chloride and potassium fluoride in an organic solvent.

7. The process of claim 6, wherein the organic solvent is acetonitrile.

8. A process for the preparation of difluoromethyl ethers of Formula (I) or pharmaceutically acceptable salts, solvates and/or prodrug thereof: 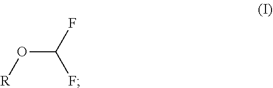
the process comprising:
a) reacting a compound of Formula (II) with Vilsmeier reagent followed by a sulfurating reagent under conditions to provide the compound of Formula (III): 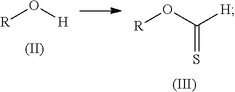
b) reacting a compound of Formula (III) with 2,2-difluoro-1,3-dimethylimidazolidine under conditions to provide the compound of Formula (I): 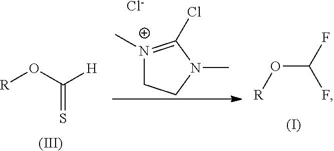
wherein
R is selected from D/L-amino acids, C₁₋₁₀alkyl, C₂₋₁₀alkenyl, C₂₋₁₀alkynyl, C₁₋₁₀haloalkyl, C₁₋₁₀cyanoalkyl, C₁₋₁₀alkoxy, C₂₋₁₀alkenyloxy, C₂₋₁₀alkynyloxy, C₃₋₁₀cycloalkyl, heterocycloalkyl, aryl, heteroaryl, C₁₋₆alkylene-O—C₁₋₆alkyl, C₁₋₆alkylene-O—C₁₋₆haloalkyl, C₂₋₆alkenylene-O—C₁₋₆haloalkyl, C₂₋₆alkynylene-O—C₁₋₆haloalkyl, C₁₋₆alkylene-C₃₋₈cycloalkyl, C₁₋₆alkylene-heterocycloalkyl, C₁₋₆alkylene-aryl, C₁₋₆alkylene-heteroaryl, C₁₋₁₀alkyl-C(O)R¹, C₂₋₁₀alkenyl-C(O)R¹, C₂₋₁₀alkynyl-C(O)R¹, C₁₋₁₀haloalkyl-C(O)R¹, C₁₋₁₀cyanoalkyl-C(O)R¹, C₁₋₁₀alkoxy-C(O)R¹, C₂₋₁₀alkenyloxy-C(O)R¹, C₃₋₁₀cycloalkyl-C(O)R¹, heterocycloalkyl-C(O)R¹, aryl-C(O)R¹, heteroaryl-C(O)R¹, C₁₋₆alkylene-O—C₁₋₆alkyl-C(O)R¹, C₁₋₆alkylene-O—C₁₋₆haloalkyl-C(O)R¹, C₂₋₆alkenylene-O—C₁₋₆haloalkyl-C(O)R¹, C₂₋₆alkenylene-O—C₁₋₆haloalkyl-C(O)R¹, C₁₋₆alkylene-C₃₋₈cycloalkyl-C(O)R¹, C₁₋₆alkylene-heterocycloalkyl-C(O)R¹, C₁₋₆alkylene-aryl-C(O)R¹, C₁₋₆alkylene-heteroaryl-C(O)R¹, C₁₋₁₀alkyl-OC(O)R¹, C₂₋₁₀alkenyl-OC(O)R¹, C₂₋₁₀alkynyl-OC(O)R¹, C₁₋₁₀haloalkyl-OC(O)R¹, C₁₋₁₀cyanoalkyl-OC(O)R¹, C₁₋₁₀alkoxy-OC(O)R¹, C₂₋₁₀alkenyloxy-OC(O)R¹, C₃₋₁₀cycloalkyl-OC(O)R¹, heterocycloalkyl-OC(O)R¹, aryl-OC(O)R¹, heteroaryl-OC(O)R¹, C₁₋₆alkylene-O—C₁₋₆alkyl-OC(O)R¹, C₁₋₆alkylene-O—C₁₋₆haloalkyl-OC(O)R¹, C₂₋₆alkenylene-O—C₁₋₆haloalkyl-O—C(O)R¹, C₂₋₆alkenylene-O—C₁₋₆haloalkyl-O—C(O)R¹, C₁₋₆alkylene-C₃₋₁₀cycloalkyl-O—C(O)R¹, C₁₋₆alkylene-heterocycloalkyl-O—C(O)R¹, C₁₋₆alkylene-aryl-O—C(O)R¹, C₁₋₆alkylene-heteroaryl-O—C(O)R¹, C₁₋₁₀alkyl-C(O)OR¹, C₂₋₁₀alkenyl-C(O)OR¹, C₂₋₁₀alkynyl-C(O)OR¹, C₁₋₁₀haloalkyl-C(O)OR¹, C₁₋₁₀cyanoalkyl-C(O)OR¹, C₁₋₁₀alkoxy-C(O)OR¹, C₂₋₁₀alkenyloxy-C(O)OR¹, C₃₋₁₀cycloalkyl-C(O)OR¹, heterocycloalkyl-C(O)OR¹, aryl-C(O)OR¹, heteroaryl-C(O)OR¹, C₁₋₆alkylene-O—C₁₋₆alkyl-C(O)OR¹, C₁₋₆alkylene-O—C₁₋₆haloalkyl-C(O)OR¹, C₂₋₆alkenylene-O—C₁₋₆haloalkyl-C(O)OR¹, C₂₋₆alkenylene-O—C₁₋₆haloalkyl-C(O)OR¹, C₁₋₆alkylene-C₃₋₈cycloalkyl-C(O)OR¹, C₁₋₆alkylene-heterocycloalkyl-C(O)OR¹, C₁₋₆alkylene-aryl-C(O)OR¹, C₁₋₆alkylene-heteroaryl-C(O)OR¹, C₁₋₆alkylene-O—R¹, C₁₋₆alkylene-C(O)R¹, C₁₋₆alkylene-O—C(O)R¹, C₁₋₆alkylene-C(O)OR¹, C₁₋₆alkylene-O—C(O)OR¹, C₁₋₆alkyleneNR¹R², C₁₋₆alkylene-NR²R¹, C₁₋₆alkylene-C(O)NR¹R², C₁₋₆alkylene-NR¹C(O)R², C₁₋₆alkylene-NR¹C(O)NR³R², C₁₋₆alkylene-S—R¹, C₁₋₆alkylene-S(O)R¹, C₁₋₆alkylene-SO₂R¹, C₁₋₆alkylene-SO₂NR¹R², C₁₋₆alkylene-NR¹SO₂R², C₁₋₆alkylene-NR³SO₂NR¹R², C(O)NR¹R² and C₁₋₆alkylene-NR¹C(O)OR², wherein R is optionally substituted with C₁₋₄alkyl and any cyclic or heterocyclic moiety is optionally fused to a further cyclic or heterocyclic moiety; and
R¹ and R² are each independently selected from the group consisting of H, C₁₋₆alkyl, C₁₋₆haloalkyl, C₂₋₆alkenyl, C₂₋₆alkynyl, C₃₋₁₀cycloalkyl, C₁₋₆alkylene-C₃₋₁₀cycloalkyl, heterocycloalkyl, aryl, C₁₋₆alkylene-aryl, C₁₋₆alkylene-heterocycloalkyl, heteroaryl, and C₁₋₆alkylene-heteroaryl, wherein any cyclic or heterocyclic moiety is optionally fused to a further cyclic or heterocyclic moiety.

9. The process of claim 8, wherein R is selected from D/L-amino acids, C₁₋₆alkyl, C₂₋₆alkenyl, C₂₋₆alkynyl, C₁₋₆haloalkyl, C₁₋₆cyanoalkyl, C₁₋₆alkoxy, C₂₋₆alkenyloxy, C₂₋₆alkynyloxy, C₃₋₆cycloalkyl, heterocycloalkyl, aryl, heteroaryl, C₁₋₄alkylene-O—C₁₋₄alkyl, C₁₋₄alkylene-O—C₁₋₄haloalkyl, C₂₋₄alkenylene-O—C₁₋₄haloalkyl, C₂₋₄alkynylene-O—C₁₋₄haloalkyl, C₁₋₄alkylene-C₃₋₆cycloalkyl, C₁₋₄alkylene-heterocycloalkyl, C₁₋₄alkylene-aryl, C₁₋₄alkylene-heteroaryl, C₁₋₆alkyl-C(O)R¹, C₂₋₆alkenyl-C(O)R¹, C₂₋₆alkynyl-C(O)R¹, C₁₋₆haloalkyl-C(O)R¹, C₁₋₆cyanoalkyl-C(O)R¹, C₁₋₆alkoxy-C(O)R¹, C₂₋₆alkenyloxy-C(O)R¹, C₃₋₆cycloalkyl-C(O)R¹, heterocycloalkyl-C(O)R¹, aryl-C(O)R¹, heteroaryl-C(O)R¹, C₁₋₄alkylene-O—C₁₋₄alkyl-C(O)R¹, C₁₋₄alkylene-O—C₁₋₄haloalkyl-C(O)R¹, C₂₋₄alkenylene-O—C₁₋₄haloalkyl-C(O)R¹, C₂₋₄alkenylene-O—C₁₋₄haloalkyl-C(O)R¹, C₁₋₄alkylene-C₃₋₆cycloalkyl-C(O)R¹, C₁₋₄alkylene-heterocycloalkyl-C(O)R¹, C₁₋₄alkylene-aryl-C(O)R¹, C₁₋₄alkylene-heteroaryl-C(O)R¹, C₁₋₆alkyl-OC(O)R¹, C₂₋₆alkenyl-OC(O)R¹, C₂₋₆alkynyl-OC(O)R¹, C₁₋₆haloalkyl-OC(O)R¹, C₁₋₆cyanoalkyl-OC(O)R¹, C₁₋₆alkoxy-OC(O)R¹, C₂₋₆alkenyloxy-OC(O)R¹, C₃₋₆cycloalkyl-OC(O)R¹, heterocycloalkyl-OC(O)R¹, aryl-OC(O)R¹, heteroaryl-OC(O)R¹, C₁₋₄alkylene-O—C₁₋₄alkyl-OC(O)R¹, C₁₋₄alkylene-O—C₁₋₄haloalkyl-OC(O)R¹, C₂₋₄alkenylene-O—C₁₋₄haloalkyl-O—C(O)R¹, C₂₋₄alkenylene-O—C₁₋₄haloalkyl-O—C(O)R¹, C₁₋₄alkylene-C₃₋₆cycloalkyl-O—C(O)R¹, C₁₋₄alkylene-heterocycloalkyl-O—C(O)R¹, C₁₋₄alkylene-aryl-O—C(O)R¹, C₁₋₄alkylene-heteroaryl-O—C(O)R¹, C₁₋₆alkyl-C(O)OR¹, C₂₋₆alkenyl-C(O)OR¹, C₂₋₆alkynyl-C(O)OR¹, C₁₋₆haloalkyl-C(O)OR¹, C₁₋₆cyanoalkyl-C(O)OR¹, C₁₋₆alkoxy-C(O)OR¹, C₂₋₆alkenyloxy-C(O)OR¹, C₃₋₆cycloalkyl-C(O)OR¹, heterocycloalkyl-C(O)OR¹, aryl-C(O)OR, heteroaryl-C(O)OR¹, C₁₋₄alkylene-O—C₁₋₄alkyl-C(O)OR¹, C₁₋₄alkylene-O—C₁₋₄haloalkyl-C(O)OR¹, C₂₋₄alkenylene-O—C₁₋₄haloalkyl-C(O)OR¹, C₂₋₄alkenylene-O—C₁₋₄haloalkyl-C(O)OR¹, C₁₋₄alkylene-C₃₋₆cycloalkyl-C(O)OR¹, C₁₋₄alkylene-heterocycloalkyl-C(O)OR¹, C₁₋₄alkylene-aryl-C(O)OR¹, C₁₋₄alkylene-heteroaryl-C(O)OR¹, C₁₋₄alkylene-O—R¹, C₁₋₄alkylene-C(O)R¹, C₁₋₄alkylene-O—C(O)R¹, C₁₋₄alkylene-C(O)OR¹, C₁₋₄alkylene-O—C(O)OR¹, C₁₋₄alkyleneNR¹R², C₁₋₄alkylene-NR²R¹, C₁₋₄alkylene-C(O)NR¹R², C₁₋₄alkylene-NR¹C(O)R², C₁₋₄alkylene-NR¹C(O)NR³R², C₁₋₄alkylene-S—R¹, C₁₋₄alkylene-S(O)R¹, C₁₋₄alkylene-SO₂R¹, C₁₋₄alkylene-SO₂NR¹R², C₁₋₄alkylene-NR¹SO₂R², C₁₋₄alkylene-NR³SO₂NR¹R², C(O)NR¹R² and C₁₋₄alkylene-NR¹C(O)OR², wherein R is optionally substituted with C₁₋₄alkyl and any cyclic or heterocyclic moiety is optionally fused to a further cyclic or heterocyclic moiety.

10. The process of claim 9, wherein R is selected from D/L-amino acids and C₁₋₆alkylene-NR¹R².

11. The process of claim 10, wherein the D/L amino acids are selected from serine and threonine.

12. The process of claim 10, wherein R is C₁₋₄alkylene-NR¹R².

13. The process of claim 8, wherein R¹ and R² are each independently selected from the group consisting of H, C₁₋₄alkyl, C₁₋₄haloalkyl, C₂₋₄alkenyl, C₂₋₄alkynyl, C₃₋₆cycloalkyl, C₁₋₄alkylene-C₃₋₆cycloalkyl, heterocycloalkyl, aryl, C₁₋₄alkylene-aryl, C₁₋₄alkylene-heterocycloalkyl, heteroaryl, and C₁₋₄alkylene-heteroaryl, wherein any cyclic or heterocyclic moiety is optionally fused to a further cyclic or heterocyclic moiety.

14. The process of claim 13, wherein R¹ and R² are selected from H and C₁₋₄alkyl.

15. The process of claim 8, wherein the Vilsmeier reagent is generated in situ from DMF and oxalyl chloride.

16. The process of claim 8, wherein the sulfurating reagent comprises hydrogen sulfide (H₂S) in the presence of pyridine or is NaSH.

17. The process of claim 16, wherein the 2,2-difluoro-1,3-dimethylimidazoline is generated in situ from 2-chloro-1,3-dimethyl-4,5-dihydroimidazol-1-ium chloride and potassium fluoride in an organic solvent.

18. The process of claim 17, wherein the organic solvent is acetonitrile.

19. The process of claim 8, wherein the compound of Formula (I) is selected from: 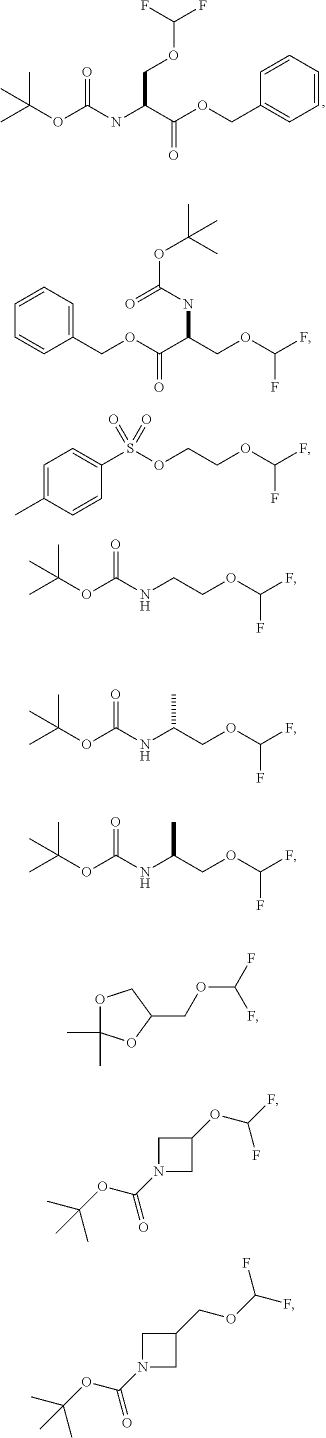 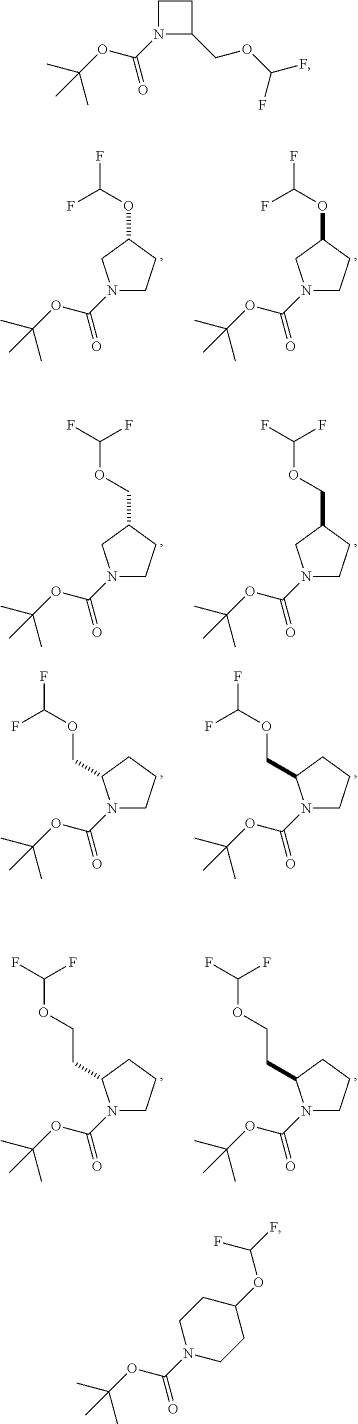 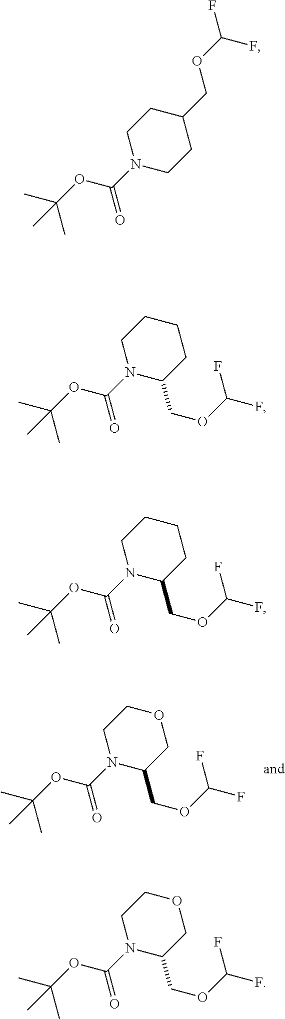 [SMILES: CC(C)(C)OC(=O)N1CCC(COC(F)F)CC1 ; CC(C)(C)OC(=O)N1CCCC[C@@H]1COC(F)F ; CC(C)(C)OC(=O)N1CCCC[C@H]1COC(F)F ; CC(C)(C)OC(=O)N1CCOC[C@H]1COC(F)F ; CC(C)(C)OC(=O)N1CCOC[C@@H]1COC(F)F]
